SEQUENCE LISTING (3 sequences)

SEQ ID NO: 1 - protein, 87 aa, Artificial
  Mouse betacellulin peptide (asp32-gln118)
DGNTTRTPETNGSLCGAPGENCTGTTPRQKVKTHFSRCPKQYKHYCIHGRCRFVVDEQTPSCICEKGYFGARCERVDLFYLQQDRGQ

SEQ ID NO: 2 - protein, 75 aa, Artificial
  NODDD sequence from Hif1a (amino acids 343-417)
SGIIQHDLIFSLQQTECVLKPVESSDMKMTQLFTKVESEDTSSLFDKLKKEPDALTLLAPAAGDTIISLDFGSND

SEQ ID NO: 3 - protein, 34 aa, Artificial
  CODDD sequence from Hif1a (amino acids 549-582)
PFSTQDTDLDLEMLAPYIPMDDDFQLRSFDQLSP

(19) United States
(12) Patent Application Publication
(10) Pub. No.: US 2006/0216295 A1
(43) Pub. Date: Sep. 28, 2006

(54) Methods of identifying therapeutic targets for the treatment of vulvovaginal atrophy

(75) Inventors: Judy Sue Crabtree, Collegeville, PA (US); Heather Anne Harris, Phoenixville, PA (US); Scott Alan Jelinsky, Waltham, MA (US); Xiaochun Zhang, Phoenixville, PA (US); Bryan John Peano, Norristown, PA (US)

Correspondence Address: POTTER ANDERSON & CORROON LLP/WYETH, 1313 NORTH MARKET STREET, HERCULES PLAZA, SIXTH FLOOR, WILMINGTON, DE, 19801, US

(73) Assignee: Wyeth, Madison, NJ (US)

(21) Appl. No.: 11/377,756

(22) Filed: Mar. 16, 2006

Related U.S. Application Data
(60) Provisional application No. 60/662,663, filed on Mar. 17, 2005.
(60) Provisional application No. 60/688,946, filed on Jun. 9, 2005.

(51) Int. Cl. A61K 48/00; A61K 38/17; A61K 39/395; A61K 31/44; A61K 31/325; A61K 31/198; A61K 31/22; A61K 31/12

(52) U.S. Cl. 424/145.1; 514/12; 514/44; 514/357; 514/561; 514/484; 514/534; 514/551; 514/685

(57) ABSTRACT

Disclosed herein are methods for the identification of effector molecules useful in the treatment of vulvovaginal atrophy. Methods of treating vulvovaginal atrophy comprising administering the effector molecules are also disclosed.

### CROSS-REFERENCE TO RELATED APPLICATIONS

This application claims priority from Provisional U.S. Patent Application Ser. No. 60/662,663, filed Mar. 17, 2005 and Provisional U.S. Patent Application Ser. No. 60/688,946, filed Jun. 9, 2005, both of which are incorporated herein by reference.

### FIELD OF THE INVENTION

The present invention relates to methods for identifying target molecules for vulvovaginal atrophy therapy through the use of gene screening, for example, mRNA expression profiling. Once a target molecule has been identified, effector molecules are used to modulate its activity in order to elicit positive effects in the vagina. The present invention also relates to methods for treating vulvovaginal atrophy using the target molecules directly or effector molecules identified by the disclosed methods.

### BACKGROUND OF THE INVENTION

Postmenopausal women suffer from a variety of symptoms associated with the reduction of endogenous estrogen production. Despite the effectiveness of estrogen therapy to alleviate symptoms of vulvovaginal atrophy (e.g., dryness, itching, dyspareunia), concerns about the side effects and long-term safety restrict its use. For example, estrogen therapy is contraindicated in women with a history of breast cancer. In addition, estrogens can increase the risk of endometrial cancer, gallbladder disease, and venous thromboembolisims. Therefore, a nonhormonal therapy that mimics the effects of estrogen in the vagina would be a viable alternative for treatment of vulvovaginal atrophy in postmenopausal women.

A number of vaginal changes occur in postmenopausal woman (see R W Steger and E S E Hafez, “Age associated changes in the vagina”, in The Human Vagina, edited by E S E Hafez and T N Evans, Elsevier/North-Holland Biomedical Press (1978)). These include, for example, thinning or loss of the vaginal epithelium, reduction in glycogen content and elastic fibers, as well as reduction in the number of blood vessels. In addition, fluid production during sexual stimulation is reduced.

Applicants have developed methods for identifying target molecules capable of mimicking the positive effects of estrogen therapy on the vaginal epithelium. Applicants have further developed methods for modulating the activity of these target molecules through the use of effector molecules to positively affect the vaginal epithelium.

### SUMMARY OF THE INVENTION

This invention provides a method of treating vulvovaginal atrophy comprising administering to a mammal in need thereof a therapeutically effective amount of an effector molecule which modulates the activity of HIF1α or betacellulin receptors.

Another aspect is for a method of screening for effector molecules which ameliorate vulvovaginal atrophy comprising:
(a) administering estrogen to a test subject;
(b) isolating mRNA from the vaginal cells of the test subject;
(c) comparing mRNA expression levels between the mRNA isolated in step (b) with mRNA isolated from a control subject;
(d) identifying a target mRNA based on the comparison of step (c); and
(e) identifying an effector molecule which modulates the activity of the target mRNA of step (d).

Another aspect is for a method of treating vulvovaginal atrophy comprising:
(a) administering estrogen to a test subject;
(b) isolating mRNA from the vaginal cells of the test subject;
(c) comparing mRNA expression levels between the mRNA isolated in step (b) with mRNA isolated from a control subject;
(d) identifying a target mRNA based on the comparison of step (c);
(e) identifying an effector molecule which modulates the activity of the target mRNA of step (d); and
(f) administering a therapeutically effective amount of the effector molecule of step (e) to a mammal in need of vulvovaginal atrophy treatment.

A further aspect is for a method identifying target mRNA which is regulated by estrogen comprising:
(a) administering estrogen to a test subject;
(b) isolating mRNA from the vaginal cells of the test subject;
(c) comparing mRNA expression levels between the mRNA isolated in step (b) with mRNA isolated from a control subject; and
(d) identifying a target mRNA based on the comparison of step (c).

Other objects and advantages of the present invention will become apparent to those skilled in the art upon reference to the detailed description that hereinafter follows.

### BRIEF DESCRIPTION OF THE FIGURES

FIG. 1 represents H&E stained cross-sections of rat vagina treated intravaginally with 2% carbomer vehicle, 50 nM 17β-estradiol, 10 nM recombinant mouse betacellulin, or 1 μM Mersalyl (a HIF1α modulator). This histology demonstrates the positive effect of these compounds on the rat vagina when compared to vehicle.

FIG. 2 represents the amino acid sequence of mouse betacellulin used as an effector molecule (Asp32-Gln118) generated in E. coli by R&D Systems (catalog # 1025-CE; Minneapolis, Minn.).

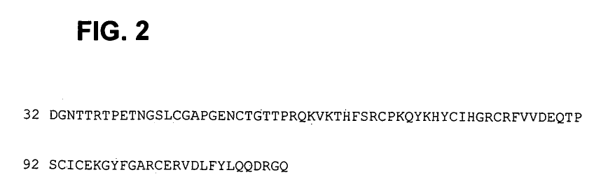

FIG. 3 represents peptide sequences for NODDD and CODDD. NODDD corresponds to amino acids 343417 of HIF1α and CODDD corresponds to amino acids 549-582 of HIF1α (William C, Masson N, Tian Y-M, Mahmood S A, Wilson M I, Bicknell R, Eckardt K-U, Maxwell P H, Ratcliffe P J, and Pugh C W. Peptide blockage of HIF1α degradation modulates cellular metabolism and angiogenesis. Proceedings of the National Academy of Sciences USA 99(16):10423-10428 (2002)).

FIG. 4 represents H&E stained cross-sections of rat vagina after 5 days of intravaginal treatment with 100 nM betacellulin in 2% carbomer vehicle. This histology demonstrates the abnormal epithelial proliferation that results in the rat vagina following intravaginal administration of betacellulin.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In the context of this disclosure, a number of terms shall be utilized.

The terms “hypoxia inducible factor 1 alpha”, “HIF1α”, and “HIF1a” refer to a protein which plays a critical role in cellular oxygen homeostasis by upregulating transcription of a wide variety of genes in response to hypoxia. HIF1α is encoded by genes such as, for example, GenBank Nos. NM_{—}181054 (human), NM_{—}001530 (human), NM_{—}024359 (rat), NM_{—}010431 (mouse), BC012527 (human), BC026139 (mouse), AF057308 (rat), AH006789 (mouse), AF004141-AF004155 (mouse), AB073325 (human), AF304431 (human), AF208487 (human), AH006957 (human), AF050127-AF050115 (human), AF003695 (mouse), Y13656 (mouse), and Y09085-Y09086 (mouse).

In the HIF1α pathway, there are several possible points of therapeutic intervention. First, HIF1α activity or protein levels can be increased by using small molecules to disrupt the rapid degradation of HIF1α (Hewitson, K S and Schofield, C J. The HIF pathway as a therapeutic target. Drug Discovery Today 9(16):704-711 (2004)). This would include, for example, inhibitors of PHD1-3 (prolyl hydroxylase domain-containing enzymes 1-3) such as, for example, oxalamic acid alkyl esters (for example, dimethyloxallyl glycine) and disubstituted pyridines (for example, diethylpyridine dicarboxylate); inhibitors of FIH (factor inhibiting HIF) such as, for example, dihydrobenzoic acids (for example, 3,4-dihydrobenzoate); proteasomal inhibitors that affect degradation of the HIF1α subunit; small molecules or antibodies that would block the von Hippel-Lindau (VHL) complex:HIF1α interaction; small molecule inhibitors of ubiquitination; and small interfering RNAs (siRNAs) targeting PHD1-3 and/or FIH.

Other useful PHD inhibitors include, for example, the nitrogen-containing heteroaryl compounds disclosed in published U.S. Patent Application No. 2004/0254215 (WO 2004/4108681), -incorporated herein by reference in its entirety. Compounds disclosed in U.S. Patent Application No. 2004/0254215 can be represented by formula I:

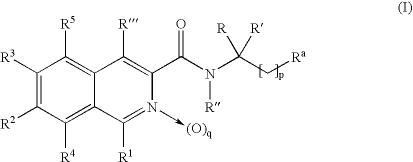

wherein:

q is zero or one;

p is zero or one;

R^{a} is —COOH or —WR⁸; provided that when R^{a} is —COOH then p is zero and when R^{a} is —WR⁸ then p is one;

W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R⁸ is not hydrogen;

R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —$(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R² and R³ are independently selected from the group consisting of hydrogen, alkyl, substituted alkyl, aryl, substituted aryl, heteroaryl, substituted heteroaryl, halo, hydroxy, cyano, —S(O)ₙ—(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R⁶ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;

R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R is selected from the group consisting of hydrogen, deuterium and methyl;

R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;

R″ is selected from the group consisting of hydrogen and alkyl or R″ together with R′ and the nitrogen pendent thereto can be joined to form a heterocyclic or substituted heterocyclic group;

R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O), —R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;

and pharmaceutically acceptable salts, esters and prodrugs thereof;

with the proviso that when R, R′ and R″ are hydrogen and q is zero, and R′ is either —COOH (p is zero) or —WR⁸ (p is one) and W is oxygen and R⁸ is hydrogen then at least one of the following occurs:

1) R′ is fluoro, bromo, iodo, alkyl, substituted alkyl, alkoxy, aminoacyl, substituted alkoxy, aryl, substituted aryl, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; or

2) R² is substituted alkyl, aryl, substituted aryl, heteroaryl, substituted heteroaryl, fluoro, bromo, iodo, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R² is substituted alkyl such a substituent does not include trifluoromethyl;

b) —XR⁶ is not alkoxy; and

c) when —XR⁶ is substituted alkoxy such a substituent does not include benzyl or benzyl substituted by a substituent selected from the group consisting of (C₁-C₅)-alkyl and (C₁-C₅)-alkoxy or does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1); or

3) R³ is substituted alkyl, aryl, substituted aryl, heteroaryl, substituted heteroaryl, bromo, iodo, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R³ is substituted alkyl such a substituent does not include trifluoromethyl;

b) —XR⁶ is not alkoxy; and

c) when —XR⁶ is substituted alkoxy such a substituent does not include benzyl or benzyl substituted by a substituent selected from the group consisting of (C₁-C₅)-alkyl and (C₁-C₅)-alkoxy or does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1); or

4) R⁴ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R⁴ is substituted alkyl such a substituent does not include trifluoromethyl;

b) —XR⁶ is not alkoxy; and

c) when —XR⁶ is substituted alkoxy such a substituent does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1); or

5) R⁵ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R⁵ is substituted alkyl such a substituent does not include trifluoromethyl;

b) —XR⁶ is not alkoxy; and

c) when —XR⁶ is substituted alkoxy such a substituent does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1);

and with the further following proviso:

that when R¹, R³, R⁴, and R⁵ are hydrogen, then R² is not bromo.

In an alternative embodiment, the compounds of formula I are represented by formula IA:

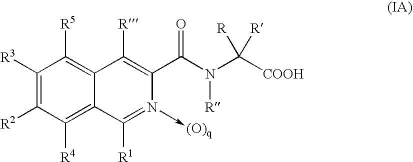

wherein R¹, R², R³, R⁴, R⁵, R, R′, R″, R′″ and q are as defined above; and

pharmaceutically acceptable salts, esters, prodrugs thereof.

In an another alternative embodiment, the compounds of formula I are represented by the formula IB:

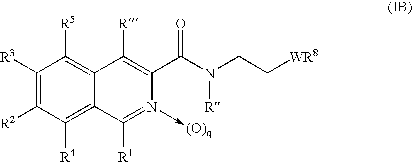

wherein R¹, R², R³, R⁴, R⁵, R″, R′″, WR⁸ and q are as defined above; and

pharmaceutically acceptable salts, esters, prodrugs thereof.

In an another alternative embodiment, the invention is directed to compounds represented by the formula IC:

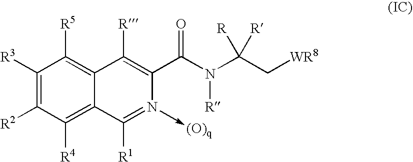

wherein R¹, R², R³, R⁴, R⁵, R, R′, R″, R′″, WR⁸ and q are as defined above; and
pharmaceutically acceptable salts, esters, prodrugs thereof.

In yet another alternative embodiment, the invention is directed to compounds represented by the formula ID:

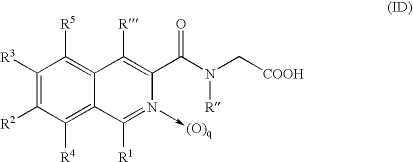

wherein R¹, R², R³, R⁴, R⁵, R, R′, R″, R′″ and q are as defined above;

and pharmaceutically acceptable salts, esters, prodrugs thereof.

Exemplary compounds disclosed in U.S. Patent Application No. 2004/0254215 include {[4-Hydroxy-1-(naphthalen-2-yloxy)-isoquinoline-3-carbonyl]-amino}-acetic acid; {[4-Hydroxy-1-(pyridin-3-yloxy)-isoquinoline-3-carbonyl]-amino}-acetic acid; {[4-Hydroxy-1-(4-methoxy-phenoxy) isoquinoline-3-carbonyl]-amino-}-acetic acid; {[4-Hydroxy-1-(3-methoxy-phenoxy)-isoquinoline-3-carbonyl]-amino}-acetic acid; {[1-(3-Fluoro-phenoxy)-4-hydroxy-isoquinoline-3-carbonyl]-amino}-acetic acid; {[1-(4-Fluoro-phenoxy)-4-hydroxy-isoquinoline-3-carbonyl]-amino}-acetic acid; {[1-(2-Fluoro-phenoxy)-4-hydroxy-isoquinoline-3-carbonyl]-amino}-acetic acid; {[4-Hydroxy-1-(2-methoxy-phenoxy)-isoquinoline-3-carbonyl]-amino}-acetic acid; {[1-(4-Acetylamino-phenoxy)-4-hydroxy-isoquinoline-3-carbonyl]-amino}-acetic acid; {[4-Hydroxy-1-(4-methanesulfonylamino-phenoxy)-isoquinoline-3-carbonyl]-amino}-acetic acid; [(4-Hydroxy-1-phenylamino-isoquinoline-3-carbonyl)-amino]-acetic acid; {[4-Hydroxy-6-(pyridin-3-yloxy)-isoquinoline-3-carbonyl]-amino}-acetic acid; {[4-Hydroxy-7-(pyridin-3-yloxy)-isoquinoline-3-carbonyl]-amino}-acetic acid; [(1-Chloro-4-methoxy-isoquinoline-3-carbonyl)-amino]-acetic acid; [(1-Chloro-4-ethoxy-isoquinoline-3-carbonyl)-amino]-acetic acid; [(4-Hydroxy-1-methoxy-isoquinoline-3-carbonyl)-amino]-acetic acid; [(1-Ethoxy-4-hydroxy-isoquinoline-3-carbonyl)-amino]-acetic acid; [(4-Acetoxy-1-phenyl-isoquinoline-3-carbonyl)-amino]-acetic acid; [(4-Hydroxy-1-phenyl-isoquinoline-3-carbonyl)-amino]-acetic acid; [(1-Ethoxy-4-phenyl-isoquinoline-3-carbonyl)-amino]-acetic acid; [(1-Chloro-4-phenyl-isoquinoline-3-carbonyl)-amino]-acetic acid; [(4-Phenyl-isoquinoline-3-carbonyl)-amino]-acetic acid; [(4-Hydroxy-1-methyl-isoquinoline-3-carbonyl)-amino]-acetic acid; [(4-Hydroxy-1-methoxymethyl-isoquinoline-3-carbonyl)-amino]-acetic acid; [(1-Dimethylcarbamoyl-4-hydroxy-isoquinoline-3-carbonyl)-amino]-acetic acid; [(4-Hydroxy-1-methyl-6-phenoxy-isoquinoline-3-carbonyl)-amino]-acetic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino]-acetic acid; [(4-Ethoxy-1-methyl-7-phenoxy-isoquinoline-3-carbonyl)-amino]-acetic acid; [(1-Dimethylcarbamoyl-4-hydroxy-7-phenoxy-isoquinoline-3-carbonyl)-amino]-acetic acid; [(4-Hydroxy-1-methoxymethyl-7-phenoxy-isoquinoline-3-carbonyl)-amino]-acetic acid; [(4-Hydroxy-1-p-tolyl-isoquinoline-3-carbonyl)-amino]-acetic acid; {[7-(4-Fluoro-phenoxy)-4-hydroxy-1-methyl-isoquinoline-3-carbonyl]-amino}-acetic acid; {[1-Chloro-4-hydroxy-7-(4-methoxy-phenoxy)-isoquinoline-3-carbonyl]-amino}-acetic acid; {[4-Hydroxy-7-(4-methoxy-phenoxy)-isoquinoline-3-carbonyl]-amino}-acetic acid; {[1-Chloro-4-hydroxy-6-(4-methoxy-phenoxy)-isoquinoline-3-carbonyl]-amino}-acetic acid; {[4-Hydroxy-6-(4-methoxy-phenoxy)-isoquinoline-3-carbonyl]-amino}-acetic acid; {[1-Chloro-4-hydroxy-7-(4-trifluoromethyl-phenoxy)-isoquinoline-3-carbonyl]-amino}-acetic acid; {[4-Hydroxy-7-(4-trifluoromethyl-phenoxy)-isoquinoline-3-carbonyl]-amino}-acetic acid; {[1-Chloro-4-hydroxy-6-(4-trifluoromethyl-phenoxy)-isoquinoline-3-carbonyl]-amino}-acetic acid; {[4-Hydroxy-6-(4-trifluoromethyl-phenoxy)-isoquinoline-3-carbonyl]-amino}-acetic acid; {[1-Chloro-7-(4-fluoro-phenoxy)-4-hydroxy-isoquinoline-3-carbonyl]-amino) acetic acid; {[7-(4-Fluoro-phenoxy)-4-hydroxy-isoquinoline-3-carbonyl]-amino}-acetic acid; {[1-Chloro-6-(4-fluoro-phenoxy)-4-hydroxy-isoquinoline-3-carbonyl]-amino}-acetic acid; {[6-(4-Fluoro-phenoxy)-4-hydroxy-isoquinoline-3-carbonyl]-amino}-acetic acid; {[4-Hydroxy-7-(pyridin-4-ylsulfanyl)-isoquinoline-3-carbonyl]-amino}-acetic acid; {[4-Hydroxy-6-(pyridin-4-ylsulfanyl)-isoquinoline-3-carbonyl]-amino}-acetic acid; [(7-Benzenesulfinyl-4-hydroxy-isoquinoline-3-carbonyl)-amino]-acetic acid; [(7-Benzenesulfonyl-4-hydroxy-isoquinoline-3-carbonyl)-amino]-acetic acid; [(6-Benzenesulfinyl-4-hydroxy-isoquinoline-3-carbonyl)-amino]-acetic acid; [(6-Benzenesulfonyl-4-hydroxy-isoquinoline-3-carbonyl)-amino]-acetic acid; [(6-Amino-4-hydroxy-isoquinoline-3-carbonyl)-amino]-acetic acid; {[4-Hydroxy-7-(4-methoxy-benzenesulfonylamino}-isoquinoline-3-carbonyl]-amino}-acetic acid; {[4-Hydroxy-7-(3-phenyl-ureido)-isoquinoline-3-carbonyl]-amino}-acetic acid; {[4-Hydroxy-6-(3-phenyl-ureido)-isoquinoline-3-carbonyl]-amino}-acetic acid; [(4-Hydroxy-1-phenylsulfanyl-isoquinoline-3-carbonyl)-amino]-acetic acid; {[1-(4-Chloro-phenylsulfanyl)-4-hydroxy-isoquinoline-3-carbonyl]-amino}-acetic acid; [(4-Hydroxy-1-p-tolylsulfanyl-isoquinoline-3-carbonyl)-amino]-acetic acid; {[4-Hydroxy-1-(pyridin-2-ylsulfanyl)-isoquinoline-3-carbonyl]-amino}-acetic acid; {[4-Hydroxy-1-(3-methoxy-phenylsulfanyl)-isoquinoline-3-carbonyl]-amino}-acetic acid; {[4-Hydroxy-1-(2-methoxy-phenylsulfanyl)-isoquinoline-3-carbonyl]-amino}-acetic acid; {[4-Hydroxy-1-(naphthalen-2-ylsulfanyl)-isoquinoline-3-carbonyl]-amino}-acetic acid; [(1-Benzenesulfinyl-4-hydroxy-isoquinoline-3-carbonyl)-amino]-acetic acid; [(1-Benzenesulfonyl-4-hydroxy-isoquinoline-3-carbonyl)-amino]-acetic acid; {[4-Hydroxy-7-(pyridin-2-ylsulfanyl)-isoquinoline-3-carbonyl]-amino}-acetic acid; {[4-Hydroxy-6-(pyridin-2-ylsulfanyl)-isoquinoline-3-carbonyl]-amino}-acetic acid; [(1-Chloro-4-hydroxy-6,7-diphenoxy-isoquinoline-3-carbonyl)-amino]-acetic acid; [(4-Hydroxy-6,7-diphenoxy-isoquinoline-3-carbonyl)-amino]-acetic acid; ({4-Hydroxy-7-[4-(toluene-4-sulfonylamino)-phenoxy]-isoquinoline-3-carbonyl}-amino)-acetic acid; {[4-Hydroxy-7-(4-nitro-phenoxy)-isoquinoline-3-carbonyl]-amino}-acetic acid; [(4-Mercapto-7-phenoxy-isoquinoline-3-carbonyl)-amino]-acetic acid; [(4-Mercapto-7-trifluoromethyl-isoquinoline-3-carbonyl)-amino]-acetic acid; {[7-(4-Benzenesulfonylamino-phenoxy)-4-hydroxy-isoquinoline-3-carbonyl]-amino}-acetic acid; {[4-Hydroxy-7-(4-methanesulfonylamino-phenoxy)-isoquinoline-3-carbonyl]-amino}-acetic acid; {[7-(4-Chloro-phenoxy)-4-hydroxy-isoquinoline-3-carbonyl]-amino}-acetic acid; {[6-(4-Chloro-phenoxy)-4-hydroxy-isoquinoline-3-carbonyl]-amino}-acetic acid; {[6-(3-Fluoro-5-methoxy-phenoxy)-4-hydroxy-isoquinoline-3-carbonyl]-amino}-acetic acid; {[7-(3-Fluoro-5-methoxy-phenoxy)-4-hydroxy-isoquinoline-3-carbonyl]-amino}-acetic acid; {[7-(3,4-Difluoro-phenoxy)-4-hydroxy-isoquinoline-3-carbonyl]-amino}-acetic acid; {[6-(3,4-Difluoro-phenoxy)-4-hydroxy-isoquinoline-3-carbonyl]-amino}-acetic acid; {[4-Hydroxy-7-(4-trifluoromethoxy-phenoxy)-isoquinoline-3-carbonyl]-amino}-acetic acid; {[4-Hydroxy-6-(4-trifluoromethoxy-phenoxy)-isoquinoline-3-carbonyl]-amino}-acetic acid; 2-(S)-{[7-(4-Chloro-phenoxy)-4-hydroxy-isoquinoline-3-carbonyl]-amino}-propionic acid; 2-(S)-{[6-(4-Chloro-phenoxy)-4-hydroxy-isoquinoline-3-carbonyl]-amino}-propionic acid; 2-{[7-(3,4-Difluoro-phenoxy)-4-hydroxy-isoquinoline-3-carbonyl]-amino}-propionic acid; 2-(S)-[(4-Hydroxy-7-phenylsulfanyl-isoquinoline-3-carbonyl)-amino]-propionic acid; 2-(R)-[(4-Hydroxy-7-phenylsulfanyl-isoquinoline-3-carbonyl)-amino]-propionic acid; 2-(R)-[(4-Hydroxy-7-phenoxy-isoquinoline-3-carbonyl)-amino]-propionic acid; 2-(S)-[4-Hydroxy-{(4-methoxy-phenoxy)-isoquinoline-3-carbonyl]-amino}-propionic acid; 2-(S)-[(7-Benzenesulfonyl-4-hydroxy-isoquinoline-3-carbonyl)-amino]-propionic acid; (R)-2-[(4-Hydroxy-1-methoxymethyl-7-phenoxy-isoquinoline-3-carbonyl)-amino]-propionic acid; (S)-2-[(4-Hydroxy-1-methoxymethyl-7-phenoxy-isoquinoline-3-carbonyl)-amino]-propionic acid; (S)-2-[(4-Mercapto-7-phenoxy-isoquinoline-3-carbonyl)-amino]-propionic acid; (S)-2-{[1-(4-Chloro-phenylsulfanyl)-4-hydroxy-isoquinoline-3-carbonyl]-amino}-propionic acid; (R)-2-{[1-(4-Chloro-phenylsulfanyl)-4-hydroxy-isoquinoline-3-carbonyl]-amino}-propionic acid; [(4-Hydroxy-7-phenylsulfanyl-isoquinoline-3-carbonyl)-amino]-acetic acid; [(4-Hydroxy-6-phenylsulfanyl-isoquinoline-3-carbonyl)-amino]-acetic acid; [(1-Chloro-4-hydroxy-7-phenylsulfanyl-isoquinoline-3-carbonyl)-amino]-acetic acid; [(1-Chloro-4-hydroxy-6-phenylsulfanyl-isoquinoline-3-carbonyl)-amino]-acetic acid; [(1-Bromo-4-hydroxy-7-phenylsulfanyl-isoquinoline-3-carbonyl)-amino]-acetic acid; [(1-Bromo-4-hydroxy-6-phenylsulfanyl-isoquinoline-3-carbonyl)-amino]-acetic acid; [(4-Hydroxy-7-phenoxy-isoquinoline-3-carbonyl)-amino]-acetic acid; [(4-Hydroxy-6-phenoxy-isoquinoline-3-carbonyl)-amino]-acetic acid; [(1-Chloro-4-hydroxy-7-phenoxy-isoquinoline-3-carbonyl)-amino]-acetic acid; [(1-Chloro-4-hydroxy-6-phenoxy-isoquinoline-3-carbonyl)-amino]-acetic acid; [(1-Bromo-4-hydroxy-7-phenoxy-isoquinoline-3-carbonyl)-amino]-acetic acid; [(1-Bromo-4-hydroxy-6-phenoxy-isoquinoline-3-carbonyl)-amino]-acetic acid; {[7-(2,6-Dimethyl-phenoxy)-4-hydroxy-isoquinoline-3-carbonyl]-amino}-acetic acid; {[1-Chloro-7-(2,6-dimethyl-phenoxy)-4-hydroxy-isoquinoline-3-carbonyl]-amino}-acetic acid; {[1-Bromo-7-(2,6-dimethyl-phenoxy)-4-hydroxy-isoquinoline-3-carbonyl]-amino}-acetic acid; [(1-Bromo-7-chloro-4-hydroxy-isoquinoline-3-carbonyl)-amino]-acetic acid; [(1-Bromo-6-chloro-4-hydroxy-isoquinoline-3-carbonyl)-amino]-acetic acid; [(1-Bromo-4-hydroxy-7-trifluoromethyl-isoquinoline-3-carbonyl)-amino]-acetic acid; [(1-Bromo-4-hydroxy-6-trifluoromethyl-isoquinoline-3-carbonyl)-amino]-acetic acid; [(4-Hydroxy-1-phenoxy-isoquinoline-3-carbonyl)-amino]-acetic acid; [(1,7-dibromo-4-hydroxy-isoquinoline-3-carbonyl)-amino]-acetic acid; [(7-Bromo-1-chloro-4-hydroxy-isoquinoline-3-carbonyl)-amino]-acetic acid; [(6-Bromo-4-hydroxy-isoquinoline-3-carbonyl)-amino]-acetic acid; [(1-Bromo-7-fluoro-4-hydroxy-isoquinoline-3-carbonyl)-amino]-acetic acid; [(7-Fluoro-4-hydroxy-isoquinoline-3-carbonyl)-amino]-acetic acid; [(1-Chloro-7-fluoro-4-hydroxy-isoquinoline-3-carbonyl)-amino]-acetic acid; [(1-Chloro-4-hydroxy-benzo[g]isoquinoline-3-carbonyl)-amino]-acetic acid; [(1-Bromo-4-hydroxy-isoquinoline-3-carbonyl)-amino]-acetic acid; [(4-Hydroxy-6-phenyl-isoquinoline-3-carbonyl)-amino]-acetic acid; [(4-Hydroxy-7-phenyl-isoquinoline-3-carbonyl)-amino]-acetic acid; [(1-Chloro-4-hydroxy-6-phenyl-isoquinoline-3-carbonyl)-amino]-acetic acid; [(1-Chloro-4-hydroxy-7-phenyl-isoquinoline-3-carbonyl)-amino]-acetic acid; [(1-Bromo-4-hydroxy-6-phenyl-isoquinoline-3-carbonyl)-amino]-acetic acid; [(1-Bromo-4-hydroxy-7-phenyl-isoquinoline-3-carbonyl)-amino]-acetic acid; [(4-Hydroxy-5-phenyl-isoquinoline-3-carbonyl)-amino]-acetic acid; [(4-Hydroxy-8-phenyl-isoquinoline-3-carbonyl)-amino]-acetic acid; [(1-Chloro-4-hydroxy-5-phenyl-isoquinoline-3-carbonyl)-amino]-acetic acid; [(1-Chloro-4-hydroxy-8-phenyl-isoquinoline-3-carbonyl)-amino]-acetic acid; [(1-Bromo-4-hydroxy-5-phenyl-isoquinoline-3-carbonyl)-amino]-acetic acid; [(1-Bromo-4-hydroxy-8-phenyl-isoquinoline-3-carbonyl)-amino]-acetic acid; [(1-Ethylsulfanyl-4-hydroxy-isoquinoline-3-carbonyl)-amino]-acetic acid; {[4-Hydroxy-1-(4-methoxy-phenylsulfanyl)-isoquinoline-3-carbonyl]-amino}-acetic acid; [(1-Chloro-4-hydroxy-7-iodo-isoquinoline-3-carbonyl)-amino]-acetic acid; [(1-Chloro-4-hydroxy-6-iodo-isoquinoline-3-carbonyl)-amino]-acetic acid; [(4-Hydroxy-7-iodo-isoquinoline-3-carbonyl)-amino]-acetic acid; [(4-Bromo-4-hydroxy-7-methyl-isoquinoline-3-carbonyl)-amino]-acetic acid; [(1-Bromo-7-butoxy-4-hydroxy-isoquinoline-3-carbonyl)-amino]-acetic acid; [(1-Bromo-6-butoxy-4-hydroxy-isoquinoline-3-carbonyl)-amino]-acetic acid; [(6-Benzyloxy-6-chloro-4-hydroxy-isoquinoline-3-carbonyl)-methyl-amino]-acetic acid; [(1-chloro-4-hydroxy-isoquinoline-3-carbonyl)-methyl-amino]-acetic acid; [(1-Chloro-4-hydroxy-6-isopropoxy-isoquinoline-3-carbonyl)-methyl-amino]-acetic acid; [(1-Chloro-4-hydroxy-7-isopropoxy-isoquinoline-3-carbonyl)-methyl-amino]-acetic acid; [Carboxymethyl-(1-chloro-4-hydroxy-isoquinoline-3-carbonyl)-amino]-acetic acid; [Carboxymethyl-(1-chloro-4-hydroxy-6-isopropoxy-isoquinoline-3-carbonyl)-amino]-acetic acid; 1-Chloro-4-hydroxy-isoquinoline-3-carboxylic acid (2-amino-ethyl)-amide(trifluoro-acetic acid salt); 1-Chloro-4-hydroxy-isoquinoline-3-carboxylic acid (2-methoxy-ethyl)-amide; 1-Chloro-4-hydroxy-isoquinoline-3-carboxylic acid (2-hydroxy-ethyl)-amide; 1-Chloro-4-hydroxy-isoquinoline-3-carboxylic acid (2-dimethylamino-ethyl)-amide; 1-Chloro-4-hydroxy-isoquinoline-3-carboxylic acid (2-acetylamino-ethyl)-amide; 1-Chloro-4-hydroxy-6-isopropoxy-isoquinoline-3-carboxylic acid (2-hydroxy-ethyl)-amide; 1-Chloro-4-hydroxy-6-isopropoxy-isoquinoline-3-carboxylic acid (2-methoxy-ethyl)-amide; 1-Chloro-4-hydroxy-6-isopropoxy-isoquinoline-3-carboxylic acid (2-amino-ethyl)-amide (trifluoro-acetic acid salt); 1-Chloro-4-hydroxy-6-isopropoxy-isoquinoline-3-carboxylic acid (2-dimethylamino-ethyl)-amide; 1-Chloro-4-hydroxy-7-isopropoxy-isoquinoline-3-carboxylic acid (2-amino-ethyl)-amide (trifluoro-acetic acid salt); 1-Chloro-4-hydroxy-7-isopropoxy-isoquinoline-3-carboxylic acid (2-methoxy-ethyl)-amide; 1-Chloro-4-hydroxy-7-isopropoxy-isoquinoline-3-carboxylic acid (2-dimethylamino-ethyl)-amide; 1-Chloro-4-hydroxy-7-isopropoxy-isoquinoline-3-carboxylic acid (2-hydroxy-ethyl)-amide; (S)-2-[(6-Benzyloxy-1-chloro-4-hydroxy-isoquinoline-3-carbonyl)-amino]-propionic acid; (R)-2-[(1-Chloro-4-hydroxy-isoquinoline-3-carbonyl)-amino]-3-hydroxy-propionic acid; (S)-2-[(1-Chloro-4-hydroxy-isoquinoline-3-carbonyl)-amino]-3-hydroxy-propionic acid; (R)-2-[(1-Chloro-4-hydroxy-6-isopropoxy-isoquinoline-3-carbonyl)-amino]-3-hydroxy-propionic acid; (S)-2-[(1-Chloro-4-hydroxy-6-isopropoxy-isoquinoline-3-carbonyl)-amino]-3-hydroxy-propionic acid; (R)-2-[(1 Chloro-4-hydroxy-7-isopropoxy-isoquinoline-3-carbonyl)-amino]-3-hydroxy-propionic acid; (S)-2-[(1-Chloro-4-hydroxy-7-isopropoxy-isoquinoline-3-carbonyl)-amino]-3-hydroxy-propionic acid; 2-[(1-Chloro-4-hydroxy-isoquinoline-3-carbonyl)-amino]-2-methyl-propionic acid; 2-[(1-Chloro-4-hydroxy-6-isopropoxy-isoquinoline-3-carbonyl)-amino]-2-methyl-propionic acid; (R)-2-[(1-Chloro-4-hydroxy-isoquinoline-3-carbonyl)-amino]-3-(1H-imidazol-4-yl)-propionic acid(trifluoro-acetic acid salt); (S)-2-[(1-Chloro-4-hydroxy-isoquinoline-3-carbonyl)-amino]-3-(1H-imidazol-4-yl)-propionic acid (trifluoro-acetic acid salt); (R)-2-[(1-Chloro-4-hydroxy-isoquinoline-3-carbonyl)-amino]-3-methyl-butyric acid; (S)-2-[(1-Chloro-4-hydroxy-isoquinoline-3-carbonyl)-amino]-3-methyl-butyric acid; (R)-2-[(1-Chloro-4-hydroxy-6-isopropoxy-isoquinoline-3-carbonyl)-amino]-3-methyl-butyric acid; (S)-2-[(1-Chloro-4-hydroxy-6-isopropoxy-isoquinoline-3-carbonyl)-amino]-3-methyl-butyric acid; (R)-2-[(1-Chloro-4-hydroxy-7-isopropoxy-isoquinoline-3-carbonyl)-amino]-3-methyl-butyric acid; (S)-2-[(1-Chloro-4-hydroxy-7-isopropoxy-isoquinoline-3-carbonyl)-amino]-3-methyl-butyric acid; (S)-2-[(6-Benzyloxy-1-chloro-4-hydroxy-isoquinoline-3-carbonyl)-amino]-3-methyl-butyric acid; (R)-2-[(1-Chloro-4-hydroxy-isoquinoline-3-carbonyl)-amino]-3-phenyl-propionic acid; (S)-2-[(1-Chloro-4-hydroxy-isoquinoline-3-carbonyl)-amino]-3-phenyl-propionic acid; (R)-2-[(1-Chloro-4-hydroxy-6-isopropoxy-isoquinoline-3-carbonyl)-amino]-3-phenyl-propionic acid; (S)-2-[(1-Chloro-4-hydroxy-6-isopropoxy-isoquinoline-3-carbonyl)-amino]-3-phenyl-propionic acid; (R)-2-[(1-Chloro-4-hydroxy-7-isopropoxy-isoquinoline-3-carbonyl)-amino]-3-phenyl-propionic acid; (S)-2-[(1-Chloro-4-hydroxy-7-isopropoxy-isoquinoline-3-carbonyl)-amino]-3-phenyl-propionic acid; (R)-2-[(1-Chloro-4-hydroxy-isoquinoline-3-carbonyl)-amino]-3-(4-hydroxy-phenyl)-propionic acid; (S)-2-[(1-Chloro-4-hydroxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-3-(4-hydroxy-phenyl)-propionic acid; (S)-2-[(1-Chloro-4-hydroxy-6-isopropoxy-isoquinoline-3-carbonyl)-amino]-3-(4-hydroxy-phenyl)-propionic acid; (R)-2-[(1-Chloro-4-hydroxy-7-isopropoxy-isoquinoline-3-carbonyl)-amino]-3-(4-hydroxy-phenyl)-propionic acid; (S)-2-[(1-Chloro-4-hydroxy-7-isopropoxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-pentanoic acid; (S)-2-[(1-Chloro-4-hydroxy-6-isopropoxy-isoquinoline-3-carbonyl)-amino]-pentanoic acid; (R)-1-(1-Chloro-4-hydroxy-isoquinoline-3-carbonyl)-pyrrolidine-2-carboxylic acid; (S)-1-(1-Chloro-4-hydroxy-isoquinoline-3-carbonyl)-pyrrolidine-2-carboxylic acid; (R)-1-(1-Chloro-4-hydroxy-6-isopropoxy-isoquinoline-3-carbonyl)-pyrrolidine-2-carboxylic acid; (S)-1-(1-Chloro-4-hydroxy-6-isopropoxy-isoquinoline-3-carbonyl)-pyrrolidine-2-carboxylic acid; (R)-6-Amino-2-[(1-chloro-4-hydroxy-isoquinoline-3-carbonyl)-amino]-hexanoic acid (trifluoro-acetic acid salt); (S)-6-Amino-2-[(1-chloro-4-hydroxy-isoquinoline-3-carbonyl)-amino]-hexanoic acid (trifluoro-acetic acid salt); (R)-6-Amino-2-[(1-chloro-4-hydroxy-6-isopropoxy-isoquinoline-3-carbonyl)-amino]-hexanoic acid; trifluoroacetic acid salt; (S)-6-Amino-2-[(1-chloro-4-hydroxy-6-isopropoxy-isoquinoline-3-carbonyl)-amino]-hexanoic acid (trifluoro-acetic acid salt); (R)-6-Amino-2-[(1-chloro-4-hydroxy-7-isopropoxy-isoquinoline-3-carbonyl)-amino]-hexanoic acid; trifluoroacetic acid salt; (S)-6-Amino-2-[(1-chloro-4-hydroxy-7-isopropoxy-isoquinoline-3-carbonyl)-amino]-hexanoic acid (trifluoro-acetic acid salt); (R)-2-[(1-Chloro-4-hydroxy-isoquinoline-3-carbonyl)-amino]-succinic acid; (S)-2-[(1-Chloro-4-hydroxy-isoquinoline-3-carbonyl)-amino]-succinic acid; (R)-2-[(1-Chloro-4-hydroxy-6-isopropoxy-isoquinoline-3-carbonyl)-amino]-succinic acid; (S)-2-[(1-Chloro-4-hydroxy-6-isopropoxy-isoquinoline-3-carbonyl)-amino]-succinic acid; (R)-2-[(1-Chloro-4-hydroxy-7-isopropoxy-isoquinoline-3-carbonyl)-amino]-succinic acid; 1-[(1-Chloro-4-hydroxy-isoquinoline-3-carbonyl)-amino]-cyclopropanecarboxylic acid; 1-[(1-Chloro-4-hydroxy-6-isopropoxy-isoquinoline-3-carbonyl)-amino]-cyclopropanecarboxylic acid; Dideutero-[(1-chloro-4-hydroxy-isoquinoline-3-carbonyl)-amino]-acetic acid; (R)-2-[(6-Benzyloxy-1-chloro-4-hydroxy-isoquinoline-3-carbonyl)-amino]-propionic acid; (S)-2-[(7-Benzyloxy-1-chloro-4-hydroxy-isoquinoline-3-carbonyl)-amino]-propionic acid; (R)-2-[(7-Benzyloxy-1-chloro-4-hydroxy-ioquinoline-3-carbonyl)-amino]-propionic acid; (S)-2-[(1-Chloro-4-hydroxy-isoquinoline-3-carbonyl)-amino]-propionic acid; (R)-2-[(1-Chloro-4-hydroxy-isoquinoline-3-carbonyl)-amino]-propionic acid; (S)-2-[(6-Isopropoxy-1-chloro-4-hydroxy-isoquinoline-3-carbonyl)-amino]-propionic acid; (R)-2-[6-Isopropoxy-1-chloro-4-hydroxy-isoquinoline-3-carbonyl)-amino]-propionic acid; (S)-2-[(7-Isopropoxy-1-chloro-4-hydroxy-isoquinoline-3-carbonyl)-amino-propionic acid; (R)-2-[(7-Isopropoxy-1-chloro-4-hydroxy-isoquinoline-3-carbonyl)-amino]-propionic acid; 1-Chloro-4-hydroxy-6-isopropoxy-isoquinoline-3-carboxylic acid (2-hydroxy-1-hydroxymethyl-ethyl)-amide; 1-Chloro-4-hydroxy-7-isopropoxy-isoquinoline-3-carboxylic acid (2-hydroxy-1-hydroxymethyl-ethyl)-amide; 1-Chloro-4-hydroxy-isoquinoline-3-carboxylic acid (2-hydroxy-1-hydroxymethyl-ethyl)-amide; {[7-(3,5-Difluoro-phenoxy)-4-hydroxy-isoquinoline-3-carbonyl]-amino}-acetic acid; {[6-(3,5-Difluoro-phenoxy)-4-hydroxy-isoquinoline-3-carbonyl]-amino}-acetic acid; ({7-[4-(4-Fluoro-phenoxy)-phenoxy]-4-hydroxy-isoquinoline-3-carbonyl}-amino)-acetic acid; ({6-[4-(4-Fluoro-phenoxy)-phenoxy]-4-hydroxy-isoquinoline-3-carbonyl}-amino)-acetic acid; {[7-(3-Chloro-4-fluoro-phenoxy)-4-hydroxy-isoquinoline-3-carbonyl]-amino}-acetic acid; {[6-(3-Chloro-4-fluoro-phenoxy)-4-hydroxy-isoquinoline-3-carbonyl]-amino}-acetic acid; (S)-2-{[7-(3-Fluoro-5-methoxy-phenoxy)-4-hydroxy-isoquinoline-3-carbonyl]-amino}-propionic acid; 2-(S)-[(7-Cyclohexyloxy-4-hydroxy-isoquinoline-3-carbonyl)-amino]-propionic acid; 2-(S)-{[7-(4-Fluoro-phenoxy)-4-hydroxy-1-methyl-isoquinoline-3-carbonyl]-amino}-propionic acid; 2-(S)-{[7-(4-Fluoro-phenoxy)-4-hydroxy-isoquinoline-3-carbonyl]-amino}-propionic acid; 2-(S)-[(4-Hydroxy-1-methyl-7-phenoxy-isoquinoline-3-carbonyl)-amino]-propionic acid; 2-(S)-[(4-Hydroxy-1-methyl-7-phenylsulfanyl-isoquinoline-3-carbonyl)-amino]-propionic acid; 2-(S)-{[4-Hydroxy-7-(4-trifluoromethyl-phenoxy)-isoquinoline-3-carbonyl]-amino}-propionic acid; {[7-(4-Chloro-phenoxy)-4-hydroxy-1-methyl-isoquinoline-3-carbonyl]-amino}-acetic acid; {[6-(4-Chloro-phenoxy)-4-hydroxy-1-methyl-isoquinoline-3-carbonyl]-amino}-acetic acid; {[7-(3,5-Difluoro-phenoxy)-4-hydroxy-1-methyl-isoquinoline-3-carbonyl]-amino}-acetic acid; {[4-Hydroxy-7-(4-methoxy-phenoxy)-1-methyl-isoquinoline-3-carbonyl]-amino}-acetic acid; {[4-Hydroxy-6-(4-methoxy-phenoxy)-1-methyl-isoquinoline-3-carbonyl]-amino}-acetic acid; [(6-Cyclohexyloxy-4-hydroxy-isoquinoline-3-carbonyl)-amino]-acetic acid; [(7-Cyclohexyloxy-4-hydroxy-isoquinoline-3-carbonyl)-amino]-acetic acid; [(7-Cyclohexyloxy-4-hydroxy-1-methyl-isoquinoline-3-carbonyl)-amino]-acetic acid; [(7-Cyclohexylsulfanyl-4-hydroxy-isoquinoline-3-carbonyl)-amino]-acetic acid; [(7-Cyclohexanesulfonyl-4-hydroxy-isoquinoline-3-carbonyl)-amino]-acetic acid; [(4-Hydroxy-1-isobutyl-isoquinoline-3-carbonyl)-amino]-acetic acid; [(4-Hydroxy-1-pyridin-2-yl-isoquinoline-3-carbonyl)-amino]-acetic acid; [(1-Ethyl-4-hydroxy-7-phenoxy-isoquinoline-3-carbonyl)-amino]-acetic acid; [(1-Dimethylaminomethyl-4-hydroxy-7-phenylsulfanyl-isoquinoline-3-carbonyl)-amino]-acetic acid; [(4-Hydroxy-1-methyl-7-phenylsulfanyl-isoquinoline-3-carbonyl)-amino]-acetic acid; {[4-Hydroxy-1-methyl-7-(4-trifluoromethyl-phenoxy)-isoquinoline-3-carbonyl]-amino}-acetic acid; and pharmaceutically acceptable salts, esters and prodrugs thereof.

Other inhibitors of PHD1-3 and active-fragments include compounds detailed in, for example, WO 2005/034929, WO 2005/007192, WO 2004/108121 (published U.S. Patent Application No. 2005/020487), WO 2003/053997 (published U.S. Patent Application No. 2003/153503), and WO 2003/049686 (published U.S. Patent Application No. 2003/176317), incorporated herein by reference in their entireties.

Compounds disclosed in WO 2004/108121 (published U.S. Patent Application No. 2005/020487) can be represented by formula II: 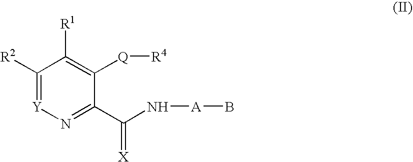
wherein

A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer.

B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO₂, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di-(C₁-C₁₂)-alkylcarbamoyl, N—(C₃-C₈)-cycloalkylcarbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₆)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N-((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C₇-C₁₆)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino-(C₁-C₈)-alkyl, amino-(C₁-C₁₀)-alkyl, N—(C₁-C₁₀)-alkylamino-(C₁-C₁₀)-alkyl, N.N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N. N-di-(C₁-C₁₂)-alkylcarbamoyl, N—(C₃-C₈)-cycloalkylcarbamoyl, N—(C₆-C₁₂)-arylcarbamoyl, N—(C₇-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₇-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, N.N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;

X is O or S;

Q is O, S, NR′, or a bond;

where, if Q is a bond, R⁴ is halogen, nitrile, or trifluoromethyl;

or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C_{f}H_{(2f+1-g)}—F_{g}—, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁)-aralkyl radical, or a radical of the formula Z
—[CH₂]ᵥ—[O]_{w}—[CH₂]ₜ-E (Z)

where

E is a heteroaryl radical, a (C₃-C₈)-cycloalkyl radical, or a phenyl radical of the formula F

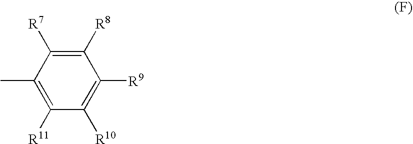

v is 0-6,

w is 0 or 1,

t is 0-3, and

R⁷, R⁸, R⁹, R¹⁰, and R″ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{Y} and R^{Z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)-aralkoxy, (C₁-C₁₂)-alkylcarbonyl, (C₃-C₈)-cycloalkylcarbonyl, (C₆-C₁₂)-arylcarbonyl, (C₇-C₁₆)-aralkylcarbonyl; or further wherein R^{Y} and R^{Z} together are —[CH₂]ₕ, in which a CH₂ group can be replaced by O, S, N—(C₁-C₄)-alkylcarbonylimino, or N—(C₁-C₄)-alkoxycarbonylimino, and h is 3 to 7; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R³ and R⁹, R⁹ and R¹⁰, or R¹⁰ and R¹¹, together are a chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷-R¹¹, or if E is a cycloalkyl radical, the radical can carry one substituent selected from those defined for R⁷-R¹¹;

or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted (C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7.

Y is N or CR³;

R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓC_{f}H_{(2f+1-g)}—F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₈)-alkoxy-(C₁-C₁₀)-alkyl)-carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R

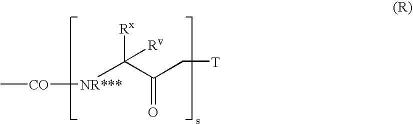

in which

R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,

s is 1-5,

T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₁-C₁₆)-aralkylimino, N—(C₁-C₄)-alkoxy-(C₁-C₆)-alkylimino, and h is from 3 to 7;

carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl) carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyl-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₁₆)-aralkyloxy-(C₁-C₁₀)-alkyl)-carbamoyl, CON(CH₂)ₕ, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy, N—(C₁-C₁₆)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy, N—(C₁-C₁₀)-alkyl-N—((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;

or wherein R¹ and R², or R² and R³ form a chain [CH₂]ₒ, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;

or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;

wherein R¹ and R², or R² and R³ form a carbocyclic or heterocyclic 5- or 6-membered aromatic ring;

or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae IIa, IIb and IIc:

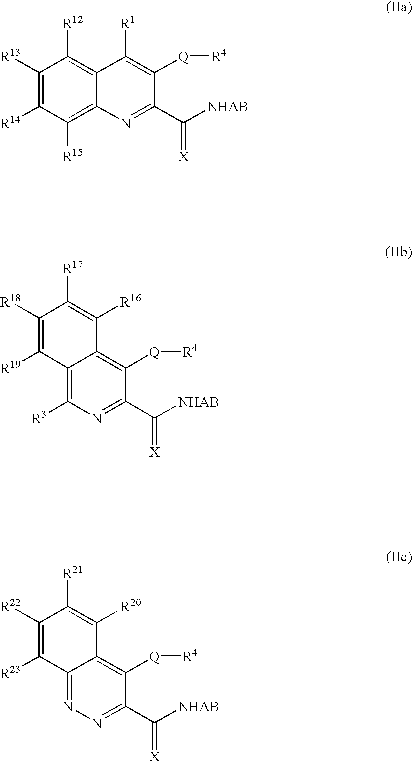

and the substituents R¹² to R²³ in each case independently of each other have the meaning of R¹, R² and R³;

or wherein the radicals R¹ and R²; together with the pyridine carrying them, form a compound of Formula IId:

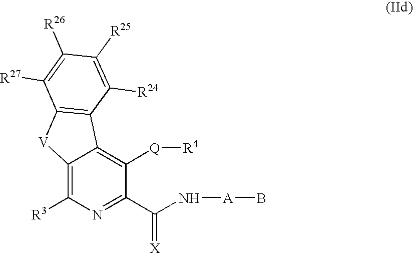

where V is S, O, or NR^{k}, and R^{k} is selected from hydrogen, (C₁-C₆)-alkyl, aryl, or benzyl;

where an aryl radical may be optionally substituted by 1 to 5 substituents as defined above; and

R²⁴, R²⁵, R²⁶, and R²⁷ in each case independently of each other have the meaning of R¹, R² and R³;

f is 1 to 8;

g is 0 or 1 to (2f+1);

x is 0 to 3; and

h is 3 to 7;

including the physiologically active salts and prodrugs derived therefrom.

Compounds disclosed in WO 2004/108121 (published U.S. Patent Application No. 2005/020487) can be represented by formula III:

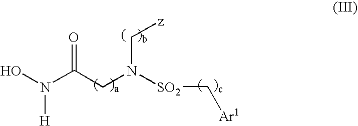

or pharmaceutically acceptable salts thereof, wherein:

a is an integer from 1 to 4;

b is an integer from 0 to 4;

c is an integer from 0 to 4;

Z is selected from the group consisting of (C₃-C₁₀)-cycloalkyl, (C₃-C₁₀)-cycloalkyl independently substituted with one or more Y¹, 3-10 membered heterocycloalkyl and 3-10 membered heterocycloalkyl independently substituted with one or more Y¹; (C₅-C₂₀)-aryl, (C₅-C₂₀)-aryl independently substituted with one or more Y¹, 5-20 membered heteroaryl and 5-20 membered heteroaryl independently substituted with one or more Y¹;

Ar¹ is selected from the group consisting of (C₅-C₂₀)-aryl, (C₅-C₂₀) aryl independently substituted with one or more Y², 5-20 membered heteroaryl and 5-20 membered heteroaryl independently substituted with one or more Y²;

each Y¹ is independently selected from the group consisting of a lipophilic functional group, (C₅-C₂₀)-aryl, (C₆-C₂₆)-alkaryl, 5-20 membered heteroaryl and 6-26 membered alk-heteroaryl;

each Y² is independently selected from the group consisting of —R′, —OR′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′)═NOR′, —NR′—C(O)R′, —SO₂R′, —SO₂R″, —NR′—SO₂—R′, —NR′—C(O)—NR′R′, tetrazol-5-yl, —NR′—C(O)—OR′, —C(NR′R′)═NR′, —S(O)—R′, S(O)—R″, and —NR′—C(S)—NR′R′; and

each R′ is independently selected from the group consisting of —H, (C₁-C₈)-alkyl, (C₂-C₈)-alkenyl, and (C₂-C₈)-alkynyl; and

each R″ is independently selected from the group consisting of (C₅-C₂₀)-aryl and (C₅-C₂₀)-aryl independently substituted with one or more OR′, —SR′, —NR′R′, —NO₂, —CN, halogen or trihalomethyl groups,

or wherein c is 0 and Ar¹ is an N′ substituted urea-aryl, the compound has the structural formula (IIIa):

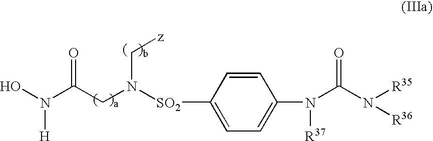

or pharmaceutically acceptable salts thereof, wherein:

a, b, and Z are as defined above; and

R³⁵ and R³⁶ are each independently selected from the group consisting of hydrogen, (C₁-C₈)-alkyl, (C₂-C₈)-alkenyl, (C₂-C₈)-alkynyl, (C₃-C₁₀)-cycloalkyl, (C₅-C₂₀)-aryl, (C₅-C₂₀)-substituted aryl, (C₆-C₂₆)-alkaryl, (C₆-C₂₆)-substituted alkaryl, 5-20 membered heteroaryl, 5-20 membered substituted heteroaryl, 6-26 membered alk-heteroaryl, and 6-26 membered substituted alk-heteroaryl; and

R³⁷ is independently selected from the group consisting of hydrogen, (C₁-C₈)-alkyl, (C₂-C₈)-alkenyl, and (C₂-C₈)-alkynyl.

Additional compounds disclosed in WO 2003/053997 (published U.S. Patent Application No. 2003/153503) can be represented by formula IV:

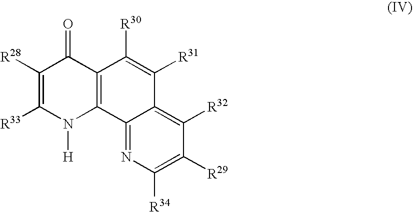

wherein

R²⁸ is hydrogen, nitro, amino, cyano, halogen, (C₁-C₄)-alkyl, carboxy or a metabolically labile ester derivative thereof; (C₁-C₄)-alkylamino, di-(C₁-C₄)-alkylamino, (C₁-C₆)-alkoxycarbonyl, (C₂-C₄)-alkanoyl, hydroxy-(C₁-C₄)-alkyl, carbamoyl, N—(C₁-C₄)-alkylcarbamoyl, (C₁-C₄)-alkylthio, (C₁-C₄)-alkylsulfinyl, (C₁-C₄)-alkylsulfonyl, phenylthio, phenylsulfinyl, phenylsulfonyl, said phenyl or phenyl groups being optionally substituted with 1 to 4 identical or different halogen, (C₁-C₄)-alkyoxy, (C₁-C₄)-alkyl, cyano, hydroxy, trifluoromethyl, fluoro-(C₁-C₄)-alkylthio, fluoro-(C₁-C₄)-alkylsulfinyl, fluoro-(C₁-C₄)-alkylsulfonyl, (C₁-C₄)-alkoxy-(C₂-C₄)-alkoxycarbonyl, N,N-di-[(C₁-C₄)-alkyl]carbamoyl-(C₁-C₄)-alkoxycarbonyl, (C₁-C₄)-alkylamino-(C₂-C₄)-alkoxycarbonyl, di-(C₁-C₄)-alkylamino-(C₂-C₄)-alkoxycarbonyl, (C₁-C₄)-alkoxy-(C₂-C₄)-alkoxy-(C₂-C₄)-alkoxycarbonyl, (C₂-C₄)-alkanoyloxy-(C₁-C₄)-alkyl, or N-[amino-(C₂-C₈)-alkyl]-carbamoyl;

R²⁹ is hydrogen, hydroxy, amino, cyano, halogen, (C₁-C₄)-alkyl, carboxy or metabolically labile ester derivative thereof, (C₁-C₄)-alkylamino, di-(C₁-C₄)-alkylamino, (C₁-C₆)-alkoxycarbonyl, (C₂-C₄)-alkanoyl, (C₁-C₄)-alkoxy, carboxy-(C₁-C₄)-alkoxy, (C₁-C₄)-alkoxycarbonyl-(C₁-C₄)-alkoxy, carbamoyl, N—(C₁-C₈)-alkylcarbamoyl, N,N-di-(C₁-C₈)-alkylcarbamoyl, N-[amino-(C₂-C₈)-alkyl]-carbamoyl, N—[(C₁-C₄)-alkylamino-(C₁-C₈)-alkyl]-carbamoyl, N-[di-(C₁-C₄)-alkylamino-(C₁-C₈)-alkyl)]-carbamoyl, N-cyclohexylcarbamoyl, N-[cyclopentyl]-carbamoyl, N—(C₁-C₄)-alkylcyclohexylcarbamoyl, N—(C₁-C₄)-alkylcyclopentylcarbamoyl, N-phenylcarbamoyl, N—(C₁-C₄)-alkyl-N-phenylcarbamoyl, N,N-diphenylcarbamoyl, N-[phenyl-(C₁-C₄)-alkyl]-carbamoyl, N—(C₁-C₄)-alkyl-N-[phenyl-(C₁-C₄)-alkyl]-carbamoyl, or N,N-di-[phenyl-(C₁-C₄)-alkyl]-carbamoyl, said phenyl or phenyl groups being optionally substituted with

1 to 4 identical or different halogen, (C₁-C₄)-alkyoxy, (C₁-C₄)-alkyl, cyano, hydroxy, trifluoromethyl, N—[(C₂-C₄)-alkanoyl]-carbamoyl, N—[(C₁-C₄)-alkoxycarbonyl]-carbamoyl, N-[fluoro-(C₂-C₆)-alkyl]-carbamoyl, N,N-[fluoro-(C₂-C₆)-alkyl]-N—(C₁-C₄)-alkylcarbamoyl, N,N-[di-fluoro-(C₂-C₆)-alkyl]carbamoyl, pyrrolidin-1-ylcarbonyl, piperidinocarbonyl, piperazin-1-ylcarbonyl, morpholinocarbonyl, wherein the heterocyclic group, is optionally substituted with 1 to 4, (C₁-C₄)-alkyl, benzyl, 1,2,3,4-tetrahydro-isoquinolin-2-ylcarbonyl, N,N-[di-(C₁-C₄)-alkyl]-thiocarbamoyl, N—(C₂-C₄)-alkanoylamino, or N—[(C₁-C₄)-alkoxycarbonyl]-amino;

R³⁰ is hydrogen, (C₁-C₄)-alkyl, (C₂-C₄)-alkoxy, halo, nitro, hydroxy, fluoro-(C₁-C₄)-alkyl, or pyridinyl;

R³¹ is hydrogen, (C₁-C₄)-alkyl, (C₂-C₄)-alkoxy, halo, nitro, hydroxy, fluoro-(C₁-C₄)-alkyl, pyridinyl, or methoxy;

R³² is hydrogen, hydroxy, amino, (C₁-C₄)-alkylamino, di-(C₁-C₄)-alkylamino, halo, (C₁-C₄)-alkoxy-(C₂-C₄)-alkoxy, fluoro-(C₁-C₆)-alkoxy, pyrrolidin-1-yl, piperidino, piperazin-1-yl, or morpholino, wherein the heterocyclic group is optionally substituted with 1 to 4 identical or different (C₁-C₄)-alkyl or benzyl; and

R³³ and R³⁴ are individually selected from hydrogen, (C₁-C₄)-alkyl, and (C₁-C₄)-alkoxy;

including pharmaceutically-acceptable salts and pro-drugs derived therefrom.

Exemplary compounds disclosed in WO 2005/034929, WO 2005/007192, WO 2004/108121 (published U.S. Patent Application No. 2005/020487), WO 2003/053997 (published U.S. Patent Application Nos. 2003/153503), and WO 2003/049686 (published U.S. Patent Application No. 2003/176317) include [(7-chloro-3-hydroxy-quinoline-2-carbonyl)-amino]-acetic acid; [(1-chloro-4-hydroxy-isoquinoline-3-carbonyl)-amino]-acetic acid; 4-oxo-1,4-dihydro-[1,10]phenathroline-3-carboxylic acid, [(3-hydroxy-6-isopropoxy-quinoline-2-carbonyl)-amino]-acetic acid; [(1-bromo-4-hydroxy-7-trifluoromethyl-isoquinoline-3-carbonyl)-amino]-acetic acid; 4-hydroxy-5-methoxy-[1,10]phenathroline-3-carboxylic acid ethyl ether; [(7-chloro-3-hydroxy-quinoline-2-carbonyl)-amino]-acetic acid, sodium salt; 3-{[4-(3,3-dibenzyl-ureido)-benzenesulfonyl]-[2-(4-methoxy-phenyl)-ethyl]-amino}-N-hydroxy-propionamide; [(4-hydroxy-7-phenylsulfanyl-isoquinoline-3-carbonyl)-amino]-acetic acid; [(4-hydroxy-6-phenylsulfanyl-isoquinoline-3-carbonyl)-amino]-acetic acid; [1-chloro-4-hydroxy-7-phenoxy-isoquinoline-3-carbonyl)-amino]-acetic acid; [(1-bromo-4-hydroxy-7-phenoxy-isoquinoline-3-carbonyl)-amino]-acetic acid; [(4-hydroxy-7-phenoxy-isoquinoline-3-carbonyl)-amino]-acetic acid; [(1-chloro-4-hydroxy-isoquinoline-3-carbonyl)-amino]-acetic acid; 3-carboxy-5-hydroxy-4-oxo-3,4-dihydro-phenathroline; 3-carboxy-5-methoxy-4-oxo-3,4-dihydro-1,10-phenathroline; 5-methoxy-4-oxo-1,4-dihydro-[1,10]phenathroline-3-carboxylic acid ethyl ester; 5-methoxy-4-oxo-1,4-dihydro-[1,10]phenanthroline-3-carboxylic acid; 3-carboxy-8-hydroxy-4-oxo-3,4-dihydro-1,10-phenanthroline; [(3-hydroxy-pyridine-2-carbonyl)-amino]-acetic acid; [(3-methoxy-pyridine-2-carbonyl)-amino]-acetic acid; 3-methoxy-pyridine-2-carboxylic acid N-(((hexadecyloxy)-carbonyl)-methyl)-amide hydrochloride; 3-methoxypyridine-2-carboxylic acid N-(((1-octyloxy)-carbonyl)-methyl)-amide; 3-methoxypyridine-2-carboxylic acid N-(((hexyloxy)-carbonyl)-methyl)-amide; 3-methoxypyridine-2-carboxylic acid N-(((butyloxy)-carbonyl)-methyl)-amide; 3-methoxypyridine-2-carboxylic acid N-(((2-nonyloxy)-carbonyl)-methyl)-amide racemate; 3-methoxypyridine-2-carboxylic acid N-(((heptyloxy)-carbonyl)-methyl)-amide; 3-benzyloxypyridine-2-carboxylic acid N-(((octyloxy)-carbonyl)-methyl)-amide; 3-benzyloxypyridine-2-carboxylic acid N-(((butyloxy)-carbonyl)-methyl)-amide; 5-(((3-(1-butyloxy)-propyl)-amino)-carbonyl)-3-methoxypyridine-2-carboxylic acid N-((benzyloxycarbonyl)-methyl)-amide; 5-(((3-(1-butyloxy)-propyl)-amino)-carbonyl)-3-methoxypyridine-2-carboxylic acid N-(((1-butyloxy)-carbonyl)-methyl)-amide; 5-(((3-lauryloxy)-propyl)amino)-carbonyl)-3-methoxypyridine-2-carboxylic acid N-(((benzyloxy)-carbonyl)-methyl)-amide; N-((6-(1-butyloxy)-3-hydroxyquinolin-2-yl)-carbonyl)-glycine; [(3-hydroxy-6-trifluoromethoxy-quinoline-2-carbonyl)-amino]-acetic acid; N-((6-chloro-3-hydroxyquinolin-2-yl)-carbonyl)-glycine; N-((7-chloro-3-hydroxyquinolin-2-yl)-carbonyl)-glycine; [(6-chloro-3-hydroxy-quinoline-2-carbonyl)-amino]-acetic acid; N-((1-chloro-4-hydroxy-7-(2-propyloxy)isoquinolin-3-yl)-carbonyl)-glycine; N-((1-chloro-4-hydroxy-6-(2-propyloxy)isoquinolin-3-yl)-carbonyl)-glycine; N-((1-chloro-4-hydroxy-isoquinoline-3-carbonyl)-amino)-acetic acid; N-((1-chloro-4-hydroxy-7-methoxyisoquinolin-3-yl)-carbonyl)-glycine; N-((1-chloro-4-hydroxy-6-methoxyisoquinolin-3-yl)-carbonyl)-glycine; N-((7-butyloxy)-1-chloro-4-hydroxyisoquinolin-3-yl)-carbonyl)-glycine; N-((6-benzyloxy-1-chloro-4-hydroxy-isoquinoline-3-carbonyl)-amino)-acetic acid; ((7-benzyloxy-1-chloro-4-hydroxy-isoquinoline-3-carbonyl)-amino)-acetic acid methyl ester; N-((7-benzyloxy-1-chloro-4-hydroxy-isoquinoline-3-carbonyl)-amino)-acetic acid; N-((8-chloro-4-hydroxyisoquinolin-3-yl)-carbonyl)-glycine; N-((7-butoxy-4-hydroxy-isoquinoline-3-carbonyl)-amino)-acetic acid; 6-cyclohexyl-1-hydroxy-4-methyl-1H-pyridin-2-one; 7-(4-methyl-piperazin-1-ylmethyl)-5-phenylsulfanylmethyl-quinolin-8-ol; 4-nitro-quinolin-8-ol; 5-butoxymethyl-quinolin-8-ol; 3-{{4-[3-(4-chloro-phenyl)-ureido]-benzenesulfonyl}-[2-(4-methoxy-phenyl)-ethyl]-amino}-N-hydroxy-propionamide; 3-{{4-[3-(1,2-diphenyl-ethyl)-ureido]-benzenesulfonyl}-[2-(4-methoxy-phenyl)-ethyl]-amino}-N-hydroxy-propionamide; and pharmaceutically acceptable salts; esters; and prodrugs thereof.

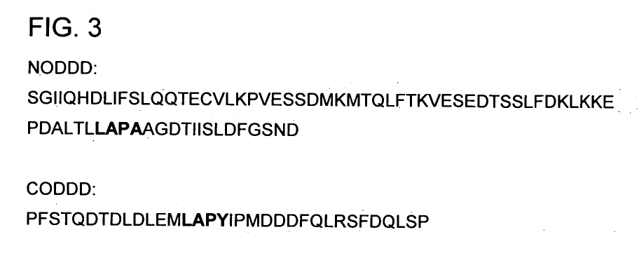

HIF1α contains an oxygen dependent degradation domain (ODDD), which has both an N-terminal portion (NODDD) and a C-terminal portion (CODDD). Hydroxylation at any of the prolyl residues in the ODDD targets the HIF1α subunit to the VHL protein for degradation; therefore, blocking the interaction of VHL with HIF1α leads to buildup of HIF1α. Also, peptides encoding the HIF1α NODDD or CODDD (see, e.g., FIG. 3) are capable of upregulating HIF-regulated transcripts in vitro (Willam C, Masson N, Tian Y-M, Mahmood S A, Wilson M I, Bicknell R, Eckardt K-U, Maxwell P H, Ratcliffe P J, and Pugh C W. Peptide blockade of HIFα degradation modulates cellular metabolism and angiogenesis. Proceedings of the National Academy of Sciences USA 99(16):10423-10428 (2002)) either by saturating the PHD enzymes or VHL binding, indicating that peptide therapy may also be efficacious.

An alternative strategy is to increase HIF1α mRNA by increasing its transcription. Compounds useful in increasing HIF1α transcription include, for example, o-substituted carbamoyl-phenoxyacetic acids. Applicants have shown that ovariectomized rats treated with mersalyl, an o-substituted carbamoyl-phenoxyacetic acid known to upregulate HIF1α transcription (Agani F and Semenza G L. Mersalyl is a novel inducer of Vascular Endothelial Growth Factor gene expression and hypoxia inducible factor 1 activity. Molecular Pharmacology 54:749-754 (1998)), show expansion of the vaginal epithelium in a manner similar to treatment with 17β-estradiol. This lends in vivo support to the notion that upregulating HIF1α leads to enhanced cellular proliferation in the rat vagina.

The term “betacellulin” refers to a member of the epidermal-growth factor (EGF) family and has been studied extensively as a diabetes therapy due to its ability to improve glucose metabolism by stimulating regeneration of pancreatic beta cells and to protect beta cells from glucose toxicity (Li L, Seno M, Yamada H, and Kojima I. Betacellulin improves glucose metabolism by promoting conversion of intraislet precursor cells to beta cells in streptozotocin-treated mice. American Journal of Physiology—Endocrinology and Metabolism. 285:E577-E583 (2003)). Betacellulin is thought to exert its activity through the EGF receptors ErbB1, ErbB2, ErbB3, and/or ErbB4, but the mechanism of betacellulin's regeneration potential is largely unknown. A similar growth promoting action of betacellulin may be at work in vaginal remodeling upon stimulation with estrogen. Other members of the family (e.g., EGF) have been demonstrated to have estrogen-like effects in the uterus and the vagina by promoting cell growth (Nelson K G, Takahashi T, Bossert N L, Walmer D K, and McLachlan J A. Epidermal growth factor replaces estrogen in the stimulation of female genital-tract growth and differentiation. Proceedings of the National Academy of Sciences USA 88:21-25 (1991)). Applicants are the first to document that betacellulin administration mimics the effects of estrogen on the rat vaginal epithelium.

Betacellulin is encoded by genes such as, for example, GenBank Nos. NM_{—}022256 (rat), NM_{—}001729 (human), NM_{—}007568 (mouse), BC011618 (human), AH011612 (mouse), AB028862 (rat), E12403 (human), S55606 (human), and L08394 (mouse).

Betacellulin can interact with all four members of the ErbB family: ErbB1-4. ErbB1 is also known as the EGFR (epidermal growth factor receptor) and ErbB2 is also known as Her2/Neu. For a review, see Alroy, I & Yarden, Y. The ErbB signaling network in embryogenesis and oncogenesis: signal diversification through combinatorial ligand-receptor interactions. FEBS Letters 410:83-86 (1997). Betacellulin has been described as a pan-ErbB ligand as it can interact/activate various receptor combinations (Dunbar A J and Goddard, C. Structure-function and biological role of betacellulin. International Journal of Biochemistry and Cell Biology 32:805-815 (2000)). Useful therapeutic strategies for mimicking betacellulin interaction with betacellulin receptors include, for example, treatment with antibodies to or small molecule agonists of betacellulin receptors.

The terms “effective amount”, “therapeutically effective amount”, and “effective dosage” as used herein, refer to the amount of an effector molecule that, when administered to a mammal in need, is effective to at least partially ameliorate a vulvovaginal atrophy condition from which the mammal is suspected to suffer. Such conditions include, but are not limited to, vaginal dryness, itching, burning, and/or tenderness; dyspareunia; recurrent urinary tract infections; and an increase in vaginal pH.

The term “effector molecule” includes, for example, agonists, partial agonists, antagonists, peptides, polypeptides, antibodies, genes, gene fragments, non-peptide small molecules, natural products, antisense DNA, antisense mRNA, siRNA, ribozymes, triplex-forming oligonucleotides, and the like.

Useful effector molecules include, for example, mersalyl; betacellulin protein, peptides, variants, or derivatives thereof; prolyl hydroxylase domain-containing enzyme (PHD) inhibitors such as, for example, DMOG (dimethyloxallyl glycine or N-(methoxyooxoacetyl)-glycine methyl ester), 2,4-DPD (2,4-diethylpyridine dicarboxylate or 2,4-pyridinedicarboxylic acid, diethyl ester), or FG-2216 (FibroGen, Inc., South San Francisco, Calif.); or factor inhibiting HIF (FIH) inhibitors such as, for example, 3,4-dihydroxybenzoate or N-oxalyl-D-alanine (NODA).

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal. The skilled person also appreciates that credible animal models of human vulvovaginal atrophy pathologies are known.

The term “modulate” encompasses either a decrease or an increase in activity depending on the target molecule. For example, an effector molecule is considered to modulate the activity of HIF1α or betacellulin if the presence of such effector molecule results in an increase in HIF1α or betacellulin mediated activity.

“Estrogen”, as used herein, includes, for example, natural estrogens, synthetic estrogens, catechol estrogens, phytoestrogens, conjugated estrogens, and non-steroidal estrogens, among others, or pharmaceutically acceptable salts or esters thereof (see, e.g., the estrogen structures described in the 11^{th} edition of “Steriods” from Steraloids, Inc., Wilton N.H.). Included in this definition are non-steroidal estrogens described in the aforementioned reference. Other estrogen compounds included in this definition are estrogen derivatives, estrogen metabolites, and estrogen precursors. Examples of estrogens having utility either alone or in combination with other agents are provided, for example, in U.S. Pat. No. 5,554,601. 17β-estradiol is a particularly preferred estrogen.

One aspect of the present invention is for a method of screening for effector molecules which ameliorate vulvovaginal atrophy comprising (a) administering estrogen to a test subject; (b) isolating mRNA from the vaginal cells of the test subject; (c) comparing mRNA expression levels between the mRNA isolated in step (b) with mRNA isolated from a control subject; (d) identifying a target mRNA based on the comparison of step (c); and (e) identifying an effector molecule which modulates the activity of the target mRNA of step (d).

Methods of administration of estrogen to a test subject are well known to those of ordinary skill in the art. For example, estrogens can be administered via pills; via gavage; transdermal patches; intravaginal gels, creams, and the like; intravginal devices; intravenously; subcutaneously; or by injection into the peritoneal cavity. Doses range from about 0.1 μg to about 100 mg, depending on the route of administration and the potency of the estrogen.

Techniques for isolating mRNA are well known to those of ordinary skill in the art. One example is provided in Jelinsky S A, Harris H A, Brown E L, Flanagan K, Zhang X, Tunkey C, Lai K, Lane M V, Simcoe D K, and Evans M J. Global transcriptional profiling of estrogen activity: Estrogen receptor a regulates gene expression in kidney. Endocrinology 144(2):701-710 (2003). Preferably, mRNA is isolated at least three hours after estrogen administration to a test subject.

In order to determine if a gene is regulated (step (c) above), the following criteria are applied: A gene is considered to be regulated if the difference between two compared groups meet the following criteria: 1) the gene is detected in at least 25% of samples of at least one of the groups, 2) the fold change between is at least 1.7, and 3) the p-value based on a T-test is ≦0.01. In a less preferred analysis, genes can also be determined to be estrogen regulated if the difference between the estrogen treated and non-treated groups meet the following criteria: 1) the gene is detected in at least 10% of the samples of at least one of the groups, 2) the fold change between the groups is at least 1.3, and 3) the p-value based on a T-test, ANOVA, Mann-Whitney Test, or Median Test is ≦0.05. Alternatively, genes can be identified through the use of only the fold change filter or through the use of only the p-value cutoff.

The test compounds of the present invention may be obtained from any available source, including systematic libraries of natural and/or synthetic compounds. Test compounds may also be obtained by any of the numerous approaches in combinatorial library-methods known in the art, including, for example, biological libraries; peptoid libraries (libraries of molecules having the functionalities of peptides, but with a novel, nonpeptide backbones that are resistant to enzymatic degradation yet remain bioactive; see, e.g., Zuckermann R N, Martin E J, Spellmeyer D C, Stauber G B, Shoemaker K R, Kerr J M, Figliozzi G M, Goff D A, Siani M A, Simon R J, Banville S C, Brown E G, Wang L, Richter L S, and Moos W H. Discovery of nanomolar ligands for 7-transmembran G-protein-coupled receptors from a diverse N-(substituted)glycine peptoid libray. Journal of Medicinal Chemistry 37:2678-85 (1994)); spatially addressable parallel solid phase or solution phase libraries; synthetic library methods requiring deconvolution; the “one-bead, one-compound” library method; and synthetic library methods using affinity chromatography selection. The biological library and peptoid library approaches are limited to peptide libraries, while the other four approaches are applicable to peptide, nonpeptide oligomer or small molecule libraries of compounds (Lam K S. Application of combinatorial library methods in cancer research and drug discovery. Anticancer Drug Design 12:145-67 (1997)).

Therapeutically suggested compounds may be provided to a mammal in need of vulvovaginal atrophy treatment in formulations that are known in the art and may include any pharmaceutically acceptable additives, such as, for example, excipents, lubricants, diluents, flavorants, colorants, and disintegrants. The formulations may be produced in useful dosage units such as, for example, tablet, caplet, capsule, liquid, or injection.

In another aspect, the invention provides prophylactic methods for preventing, in a mammal, vulvovaginal atrophy, by administering to the mammal an effector molecule, which modulates target molecule expression and/or activity. Administration of a prophylactic agent can occur prior to the manifestation of symptoms characteristic of vulvovaginal atrophy, such that vulvovaginal atrophy is prevented or, alternatively, delayed in its progression.

Preferred delivery systems include, for example, those that provide a sustained delivery of the effector molecule to the vaginal epithelium and mucosa for the treatment of vulvovaginal atrophy. A delivery system can comprise a device such as, for example, a tampon, tampon-like device, vaginal ring, pessary, cup, vaginal ring, cervical cup or vaginal sponge, containing an effector molecule in the form of a paste, cream, ointment, microcapsules, solution, powder, or gel having a sufficient viscosity to maintain prolonged vaginal epithelium and mucosa contact.

Alternatively, the effector molecule can be incorporated into a coating on a tampon or tampon-like device, sponge, suppository, or other absorbent material impregnated with a liquid, drug containing solution, lotion, or suspension of bioadhesive particles, shaped into a tampon-fitting device. Any form of effector molecule delivery system which will effectively deliver the effector molecule to the vaginal epithelium and mucosa or transvaginally through the vaginal mucosa is intended to be included within the scope of this invention.

The form and amount of therapeutic compound envisioned for use depends on the type of disease and the severity of the desired effect, patient state, etc., and can be determined by one skilled in the art.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

### Example 1

Ovariectomized rats were treated with a single dose of 20 μg/kg 17β-estradiol subcutaneously, and the vaginal vault was harvested 6 hours, 2 days and 6 hours, or 5 days and 6 hours post-treatment. RNA was harvested from the entire vaginal vault at each time point and was subjected to RNA expression analysis. Five micrograms of total RNA were used to generate biotin labeled cRNA using an oligo T7 primer in a reverse transcription reaction followed by in vitro transcription reaction with biotin labeled UTP and CTP. Ten micrograms of cRNA were fragmented and hybridized to RAE230A and RAE230B arrays (Affymetrix, Santa Clara, Calif.). Hybridized arrays were stained according to manufacturer's protocols on a Fluidics Station 450 and scanned on an Affymetrix scanner 3000. All array images were visually inspected for defects and quality. Signal values were determined using Gene Chip Operating System 1.0 (GCOS, Affymetrix). For each array, all probe sets were normalized to a mean signal intensity value of 100. The default GCOS statistical values were used for all analyses. Signal values and absolute detection calls were imported into Genesis 2.0 (GeneLogic, Gaithersburg, Md.) for analysis.

A gene was considered to be detectible if the mean expression in either the treated or untreated group was 50 signal units and the percentage of samples with a Present (P) call as determined by GCOS default settings was greater than or equal to 25%. A gene was considered to be regulated if the difference between the treated and untreated groups met the following criteria: (1) the gene had to be detected in at least 25% of the samples of at least one of the groups, (2) the fold change between was at least 1.7, and (3) the p-value based on a T-test had to be ≦0.01. Nine hundred fifty-nine qualifiers on the RAE230A array and 707 qualifiers on the RAE230B array met these conditions.

Based on the time course of response and the biological function associated with them, HIF1α and betacellulin were selected as candidate targets for treatment of vulvovaginal atrophy. The fold change of these three genes over vehicle is shown in Table 1. These proteins were selected for further study because they were regulated by estradiol after 6 hours of treatment and thus can be expected to be at the top of the estrogenic signaling cascade.

TABLE 1
Fold Change Over Vehicle

| | 6 hour | 72 hour (2 day) | 120 hour (5 day) |
|---|---|---|---|
| Betacellulin | +2.9 | +2.8 | +1.6 |
| HIF1α | +2.2 | +2.6 | +4.6 |

Proteins expressed by these genes, based on their regulation in response to estradiol are expected to participate in morphological changes in the vagina in response to estrogen and are targets for the development of small molecules or proteins that mimic their positive effects on the vaginal epithelium.

### Example 2

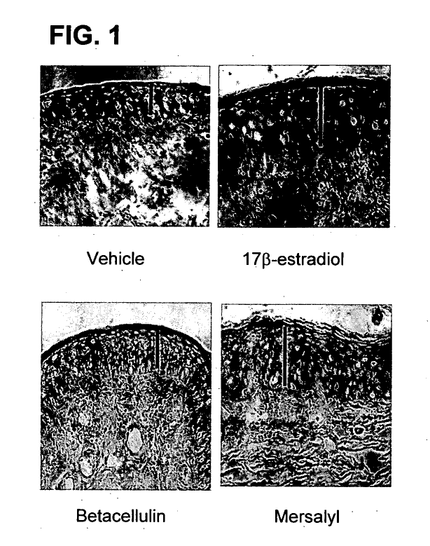

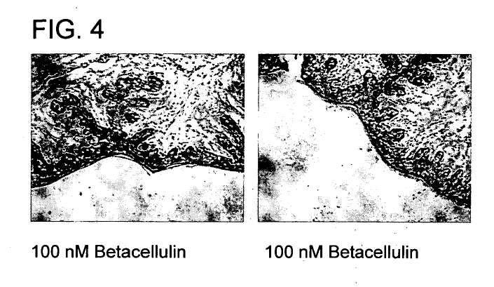

Studies in rats using intravaginal dosing of compounds known to modulate HIF1α (mersalyl) or activate betacellulin receptors (administration of betacellulin protein itself) demonstrated that modulation of each of these three are capable of mimicking the action of 17β-estradiol in the vagina. Histologically, there is a clear thickening and differentiation of the vaginal epithelial layer, and although it does not form in humans, the formation of a keratin layer when compared to vehicle (see FIG. 1). These histologic changes are the hallmark of a vaginal estrogenic response. Tables 2 and 3 detail the studies performed with mersalyl and betacellulin. The body of the table indicates the number of positive histological changes over the number of samples evaluated. In some cases of betacellulin treatment, abnormal proliferation of vaginal epithelium was noted, as in FIG. 4.

TABLE 2

| Mersalyl | | | | |
|---|---|---|---|---|
| dose | Study 1 | Study 2 | Study 3 | Study 4 |
| 0.1 μM | ND | 2/4 | 4/4 | 4/4 |
| 1 μM | 3/3 | 4/4/ | 2/3 | 4/4/ |
| 10 μM | 0/3 | 0/4 | ND | ND |
| 100 μM | 0/3 | ND | ND | ND |

TABLE 3

| Betacellulin dose | | Study 1 | Study 2 | Study 3 |
|---|---|---|---|---|
| 10 | nM | 3/3 | 0/4 | 3/3 |
| 100 | nM | ND | 3/4 | 2/3 |
| 1 | μM | Nd | 2/4 | ND |

### Example 3

Ovariectomized rats treated intravaginally with compounds known to modulate PHD (DMOG or 2,4-DPD) or FIH (3,4-dihydroxybenzoate or N-oxalyl-D-alanine) demonstrate variable results regarding whether inhibition of either of these enzymes mimics the action of 17β-estradiol in the vagina. In some cases, a clear thickening and differentiation of the vaginal epithelial layer, and although it does not form in humans, the formation of a keratin layer was present when compared to vehicle—but this response was not present in all samples.

Table 4 includes the studies performed with the PHD/FIH non-specific inhibitor DMOG, whereas Table 5 is the studies performed with 2,4-DPD. Data is presented as number of positive histological responses/number of samples evaluated and reveals variability in the histological response of these compounds in the ovariectomized (OVX) rat vagina.

Table 6 includes the studies performed with the FIH specific inhibitor 3,4-dihydroxybenzoate (3,4-DHB), whereas Table 7 outlines the studies performed with N-oxalyl-D-alanine (NODA). Data is presented as number of positive histological responses/number of samples evaluated and again, reveals variability in the histological response of these compounds in the OVX rat vagina. These results indicate the dosing regimen and/or compound may not be optimized.

TABLE 4

| DMOG | | Study 1 | Study 2 | Study 3 |
|---|---|---|---|---|
| 1 | μM | ND | 0/4 | ND |
| 10 | μM | 3/3 | 2/3 | 2/4 |
| 100 | μM | 0/3 | ND | ND |
| 1 | mM | 0/3 | ND | 1/4 |

TABLE 5

| 2,4-DPD | Study 1 | Study 2 | Study 3 |
|---|---|---|---|
| 0.1 μM | ND | 0/4 | ND |
| 1 μM | ND | 1/4 | ND |
| 10 μM | 0/4 | 1/3 | 1/4 |

TABLE 6

| 3,4-DHB | | Study 1 | Study 2 | Study 3 | Study 4 |
|---|---|---|---|---|---|
| 10 | μM | 1/4 | ND | ND | ND |
| 100 | μM | 0/4 | ND | ND | ND |
| 1 | mM | 0/4 | 0/4 | 0/3 | ND |
| 10 | mM | 4/4 | 1/4 | 2/3 | 1/3 |

TABLE 7

| NODA | | Study 1 | Study 2 | Study 3 | Study 4 |
|---|---|---|---|---|---|
| 10 | μM | 1/4 | 1/3 | 0/3 | ND |
| 100 | μM | 0/4 | 0/3 | 0/3 | ND |
| 1 | mM | 0/4 | 0/3 | 1/3 | 1/4 |
| 10 | mM | 3/4 | 0/3 | 1/3 | 0/4 |

### Example 4

Ovariectomized rats would be treated intravaginally with peptides known to compete with HIF1α (NODDD or CODDD) for PHD binding (i.e. competitive inhibitor) to demonstrate that interference with HIF1α degradation mimics the action of 17β-estradiol in the vagina. Histologically, Applicants would expect a clear thickening and differentiation of the vaginal epithelial layer, and although it does not form in humans, the formation of a keratin layer when compared to vehicle. These histological changes are the hallmark of a vaginal estrogenic response and would lend in vivo support to the notion of blocking HIF1α degradation as a way to increase HIF1α levels in the rat vagina for the treatment of vulvovaginal atrophy.

What is claimed is:

1. A method of treating vulvovaginal atrophy comprising administering to a mammal in need thereof a therapeutically effective amount of an effector molecule which modulates the activity of HIF1α or betacellulin receptors.

2. The method of claim 1, wherein the effector molecule is an agonist, partial agonist, antagonist, peptide, polypeptide, antibody, gene, gene fragment, non-peptide small molecule, natural product, antisense DNA, antisense mRNA, siRNA, ribozyme, or triplex-forming oligonucleotide.

3. The method of claim 1, wherein the effector molecule increases HIF1α activity.

4. The method of claim 3, wherein the effector molecule is selected from diphenylalkanes or o-substituted carbamoyl-phenoxyacetic acids.

5. The method of claim 4, wherein the diphenylalkane is dibenzoylmethane.

6. The method of claim 4, wherein the o-substituted carbamoyl-phenoxyacetic acid is mersalyl.

7. The method of claim 3, wherein the effector molecule is a PHD inhibitor or an FIH inhibitor.

8. The method of claim 7, wherein the PHD inhibitor is selected from oxalamic acid alkyl esters or disubstituted pyridines.

9. The method of claim 8, wherein the oxalamic acid alkyl ester is dimethyloxallyl glycine.

10. The method of claim 8, wherein the disubstituted pyridine is 2,4-diethylpyridine dicarboxylate.

11. The method of claim 7, wherein the PHD inhibitor is a nitrogen-containing heteroaryl compound.

12. The method of claim 11, wherein the nitrogen-containing heteroaryl compound is represented by formula I: 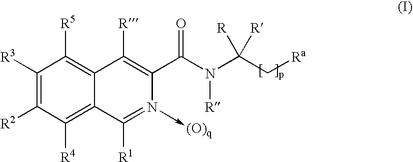
wherein:
q is zero or one;
p is zero or one;
R^{a} is —COOH or —WR⁸; provided that when R^{a} is —COOH then p is zero and when R^{a} is —WR⁸ then p is one;
W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R⁸ is not hydrogen;
R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R² and R³ are independently selected from the group consisting of hydrogen, alkyl, substituted alkyl, aryl, substituted aryl, heteroaryl, substituted heteroaryl, halo, hydroxy, cyano, —S(O)ₙ—(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R⁶ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R is selected from the group consisting of hydrogen, deuterium and methyl;
R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;
R″ is selected from the group consisting of hydrogen and alkyl or R″ together with R′ and the nitrogen pendent thereto can be joined to form a heterocyclic or substituted heterocyclic group;
R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)ₙ—R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;
and pharmaceutically acceptable salts, esters and prodrugs thereof;
with the proviso that when R, R′ and R″ are hydrogen and q is zero, and R^{a} is either —COOH (p is zero) or —WR⁸ (p is one) and W is oxygen and R⁸ is hydrogen then at least one of the following occurs:
1) R′ is fluoro, bromo, iodo, alkyl, substituted alkyl, alkoxy, aminoacyl, substituted alkoxy, aryl, substituted aryl, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; or
2) R² is substituted alkyl, aryl, substituted aryl, heteroaryl, substituted heteroaryl, fluoro, bromo, iodo, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
a) when R² is substituted alkyl such a substituent does not include trifluoromethyl;
b) —XR⁶ is not alkoxy; and
c) when —XR⁶ is substituted alkoxy such a substituent does not include benzyl or benzyl substituted by a substituent selected from the group consisting of (C₁-C₅)-alkyl and (C₁-C₅)-alkoxy or does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1); or
3) R³ is substituted alkyl, aryl, substituted aryl, heteroaryl, substituted heteroaryl, bromo, iodo, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
a) when R³ is substituted alkyl such a substituent does not include trifluoromethyl;
b) —XR⁶ is not alkoxy; and
c) when —XR⁶ is substituted alkoxy such a substituent does not include benzyl or benzyl substituted by a substituent selected from the group consisting of (C₁-C₅)-alkyl and (C₁-C₅)-alkoxy or does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1); or
4) R⁴ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
a) when R⁴ is substituted alkyl such a substituent does not include trifluoromethyl;
b) —XR⁶ is not alkoxy; and
c) when —XR⁶ is substituted alkoxy such a substituent does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1); or
5) R⁵ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
a) when R⁵ is substituted alkyl such a substituent does not include trifluoromethyl;
b) —XR⁶ is not alkoxy; and
c) when —XR⁶ is substituted alkoxy such a substituent does not include a fluoroalkoxy substituent of the formula:
—O—[CH₂]ₓ—C_{f}H_{(2f+1-g)}F_{g}
where x is zero or one; f is an integer of from 1 to 5; and g is an integer of from 1 to (2f+1);
and with the further following proviso:
that when R¹, R³, R⁴, and R⁵ are hydrogen, then R² is not bromo.

13. The method of claim 12, wherein the nitrogen-containing heteroaryl compound is represented by formula IA: 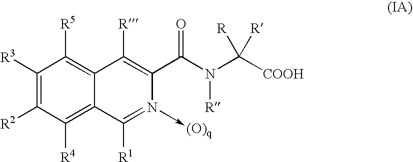
wherein R¹, R², R³, R⁴, R⁵, R, R′, R″, R′″ and q are as defined in claim 12; and
pharmaceutically acceptable salts, esters, prodrugs thereof.

14. The method of claim 12, wherein the nitrogen-containing heteroaryl compound is represented by formula IB: 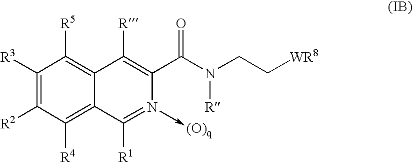
wherein R¹, R², R³, R⁴, R⁵, R″, R′″, WR⁸ and q are as in claim 12; and
pharmaceutically acceptable salts, esters, prodrugs thereof.

15. The method of claim 12, wherein the nitrogen-containing heteroaryl compound is represented by formula IC: 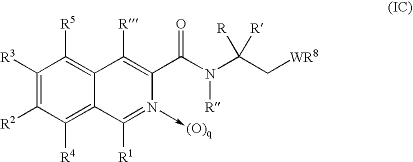
wherein R¹, R², R³, R⁴, R⁵, R′, R″, R′″, WR⁸ and q are as in claim 12; and
pharmaceutically acceptable salts, esters, prodrugs thereof.

16. The method of claim 12, wherein the nitrogen-containing heteroaryl compound is represented by formula ID: 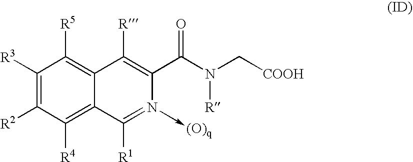
wherein R¹, R², R³, R⁴, R⁵, R, R′, R″, R′″ and q are as in claim 12; and
pharmaceutically acceptable salts, esters, prodrugs thereof.

17. The method of claim 11, wherein the nitrogen-containing heteroaryl compound is selected from {[4-Hydroxy-1-(naphthalen-2-yloxy)-isoquinoline-3-carbonyl]-amino}-acetic acid; {[4-Hydroxy-1-(pyridin-3-yloxy)-isoquinoline-3-carbonyl]-amino}-acetic acid; {[4-Hydroxy-1-(4-methoxy-phenoxy)-isoquinoline-3-carbonyl]-amino-}-acetic acid; {[4-Hydroxy-1-(3-methoxy-phenoxy)-isoquinoline-3-carbonyl]-amino)-acetic acid; {[1-(3-Fluoro-phenoxy)-hydroxy-isoquinoline-3-carbonyl]-amino}-acetic acid; {[1-(4-Fluoro-phenoxy)-4-hydroxy-isoquinoline-3-carbonyl]-amino}-acetic acid; {[1-(2-Fluoro-phenoxy)-4-hydroxy-isoquinoline-3-carbonyl]-amino}-acetic acid; {[4-Hydroxy-1-(2-methoxy-phenoxy)-isoquinoline-3-carbonyl]-amino}-acetic acid; {[1-(4-Acetylamino-phenoxy)-4-hydroxy-isoquinoline-3-carbonyl]-amino}-acetic acid; {[4-Hydroxy-1-(4-methanesulfonylamino-phenoxy)-isoquinoline-3-carbonyl]-amino}-acetic acid; [(4-Hydroxy-1-phenylamino-isoquinoline-3-carbonyl)-amino]-acetic acid; {[4-Hydroxy-6-(pyridin-3-yloxy)-isoquinoline-3-carbonyl]-amino}-acetic acid; {[4-Hydroxy-7-(pyridin-3-yloxy)-isoquinoline-3-carbonyl]-amino}-acetic acid; [(1-Chloro-4-methoxy-isoquinoline-3-carbonyl)-amino]-acetic acid; [(1-Chloro-4-ethoxy-isoquinoline-3-carbonyl)-amino]-acetic acid; [(4-Hydroxy-1-methoxy-isoquinoline-3-carbonyl)-amino]-acetic acid; [(1-Ethoxy-4-hydroxy-isoquinoline-3-carbonyl)-amino]-acetic acid; [(4-Acetoxy-1-phenyl-isoquinoline-3-carbonyl)-amino]-acetic acid; [(4-Hydroxy-1-phenyl-isoquinoline-3-carbonyl)-amino]-acetic acid; [(1-Ethoxy-4-phenyl-isoquinoline-3-carbonyl)-amino]-acetic acid; [(1-Chloro-4-phenyl-isoquinoline-3-carbonyl)-amino]-acetic acid; [(4-Phenyl-isoquinoline-3-carbonyl)-amino]-acetic acid; [(4-Hydroxy-1-methyl-isoquinoline-3-carbonyl)-amino]-acetic acid; [(4-Hydroxy-1-methoxymethyl-isoquinoline-3-carbonyl)-amino]-acetic acid; [(1-Dimethylcarbamoyl-4-hydroxy-isoquinoline-3-carbonyl)-amino]-acetic acid; [(4-Hydroxy-1-methyl-6-phenoxy-isoquinoline-3-carbonyl)-amino]-acetic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino]-acetic acid; [(4-Ethoxy-1-methyl-7-phenoxy-isoquinoline-3-carbonyl)-amino]-acetic acid; [(1-Dimethylcarbamoyl-4-hydroxy-7-phenoxy-isoquinoline-3-carbonyl)-amino]-acetic acid; [(4-Hydroxy-1-methoxymethyl-7-phenoxy-isoquinoline-3-carbonyl)-amino]-acetic acid; [(4-Hydroxy-1-p-tolyl-isoquinoline-3-carbonyl)-amino]-acetic acid; {[7-(4-Fluoro-phenoxy)-4-hydroxy-1-methyl-isoquinoline-3-carbonyl]-amino}-acetic acid; {[1-Chloro-4-hydroxy-7-(4-methoxy-phenoxy)-isoquinoline-3-carbonyl]-amino}-acetic acid; {[4-Hydroxy-7-(4-methoxy-phenoxy)-isoquinoline-3-carbonyl]-amino}-acetic acid; {[1-Chloro-4-hydroxy-6-(4-methoxy-phenoxy)-isoquinoline-3-carbonyl]-amino}-acetic acid; {[4-Hydroxy-6-(4-methoxy-phenoxy)-isoquinoline-3-carbonyl]-amino}-acetic acid; {[1-Chloro-4-hydroxy-7-(4-trifluoromethyl-phenoxy)-isoquinoline-3-carbonyl]-amino}-acetic acid; {[4-Hydroxy-7-(4-trifluoromethyl-phenoxy)-isoquinoline-3-carbonyl]-amino}-acetic acid; {[1-Chloro-4-hydroxy-6-(4-trifluoromethyl-phenoxy)-isoquinoline-3-carbonyl]-amino}-acetic acid; {[4-Hydroxy-6-(4-trifluoromethyl-phenoxy)-isoquinoline-3-carbonyl]-amino}-acetic acid; {[1-Chloro-7-(4-fluoro-phenoxy)-4-hydroxy-isoquinoline-3-carbonyl]-amino}-acetic acid; {[7-(4-Fluoro-phenoxy)-4-hydroxy-isoquinoline-3-carbonyl]-amino}-acetic acid; {[1-Chloro-6-(4-fluoro-phenoxy)-4-hydroxy-isoquinoline-3-carbonyl]-amino}-acetic acid; {[6-(4-Fluoro-phenoxy)-4-hydroxy-isoquinoline-3-carbonyl]-amino}-acetic acid; {[4-Hydroxy-7-(pyridin-4-ylsulfanyl)-isoquinoline-3-carbonyl]-amino}-acetic acid; {[4-Hydroxy-6-(pyridin-4-ylsulfanyl)-isoquinoline-3-carbonyl]-amino}-acetic acid; [(7-Benzenesulfinyl-4-hydroxy-isoquinoline-3-carbonyl)-amino]-acetic acid; [(7-Benzenesulfonyl-4-hydroxy-isoquinoline-3-carbonyl)-amino]-acetic acid; [(6-Benzenesulfinyl-4-hydroxy-isoquinoline-3-carbonyl)-amino]-acetic acid; [(6-Benzenesulfonyl-4-hydroxy-isoquinoline-3-carbonyl)-amino]-acetic acid; [(6-Amino-4-hydroxy-isoquinoline-3-carbonyl)-amino]-acetic acid; {[4-Hydroxy-7-(4-methoxy-benzenesulfonylamino}-isoquinoline-3-carbonyl]-amino}-acetic acid; {[4-Hydroxy-7-(3-phenyl-ureido)-isoquinoline-3-carbonyl]-amino}-acetic acid; {[4-Hydroxy-6-(3-phenyl-ureido)-isoquinoline-3-carbonyl]-amino}-acetic acid; [(4-Hydroxy-1-phenylsulfanyl-isoquinoline-3-carbonyl)-amino]-acetic acid; {[1-(4-Chloro-phenylsulfanyl)-4-hydroxy-isoquinoline-3-carbonyl]-amino}-acetic acid; [(4-Hydroxy-1-p-tolylsulfanyl-isoquinoline-3-carbonyl)-amino]-acetic acid; {[4-Hydroxy-1-(pyridin-2-ylsulfanyl)-isoquinoline-3-carbonyl]-amino}-acetic acid; {[4-Hydroxy-1-(3-methoxy-phenylsulfanyl)-isoquinoline-3-carbonyl]-amino}-acetic acid; {[4-Hydroxy-1-(2-methoxy-phenylsulfanyl)-isoquinoline-3-carbonyl]-amino}-acetic acid; {[4-Hydroxy-1-(naphthalen-2-ylsulfanyl)-isoquinoline-3-carbonyl]-amino}-acetic acid; [(1-Benzenesulfinyl-4-hydroxy-isoquinoline-3-carbonyl)-amino]-acetic acid; [(1-Benzenesulfonyl-4-hydroxy-isoquinoline-3-carbonyl)-amino]-acetic acid; {[4-Hydroxy-7-(pyridin-2-ylsulfanyl)-isoquinoline-3-carbonyl]-amino}-acetic acid; {[4-Hydroxy-6-(pyridin-2-ylsulfanyl)-isoquinoline-3-carbonyl]-amino}-acetic acid; [(1-Chloro-4-hydroxy-6,7-diphenoxy-isoquinoline-3-carbonyl)-amino]-acetic acid; [(4-Hydroxy-6,7-diphenoxy-isoquinoline-3-carbonyl)-amino]-acetic acid; ({4-Hydroxy-7-[4-(toluene-4-sulfonylamino)-phenoxy]-isoquinoline-3-carbonyl}-amino)-acetic acid; {[4-Hydroxy-7-(4-nitro-phenoxy)-isoquinoline-3-carbonyl]-amino}-acetic acid; [(4-Mercapto-7-phenoxy-isoquinoline-3-carbonyl)-amino]-acetic acid; [(4-Mercapto-7-trifluoromethyl-isoquinoline-3-carbonyl)-amino]-acetic acid; {[7-(4-Benzenesulfonylamino-phenoxy)-4-hydroxy-isoquinoline-3-carbonyl]-amino}-acetic acid; {[4-Hydroxy-7-(4-methanesulfonylamino-phenoxy)-isoquinoline-3-carbonyl]-amino}-acetic acid; {[7-(4-Chloro-phenoxy)-4-hydroxy-isoquinoline-3-carbonyl]-amino}-acetic acid; {[6-(4-Chloro-phenoxy)-4-hydroxy-isoquinoline-3-carbonyl]-amino}-acetic acid; {[6-(3-Fluoro-5-methoxy-phenoxy)-4-hydroxy-isoquinoline-3-carbonyl]-amino}-acetic acid; {[7-(3-Fluoro-5-methoxy-phenoxy)-4-hydroxy-isoquinoline-3-carbonyl]-amino}-acetic acid; {[7-(3,4-Difluoro-phenoxy)-hydroxy-isoquinoline-3-carbonyl]-amino}-acetic acid; {[6-(3,4-Difluoro-phenoxy)-4-hydroxy-isoquinoline-3-carbonyl]-amino}-acetic acid; {[4-Hydroxy-7-(4-trifluoromethoxy-phenoxy)-isoquinoline-3-carbonyl]-amino}-acetic acid; {[4-Hydroxy-6-(4-trifluoromethoxy-phenoxy)-isoquinoline-3-carbonyl]-amino}-acetic acid; 2-(S)-{[7-(4-Chloro-phenoxy)-4-hydroxy-isoquinoline-3-carbonyl]-amino}-propionic acid; 2-(S)-{[6-(4-Chloro-phenoxy)-4-hydroxy-isoquinoline-3-carbonyl]-amino}-propionic acid; 2-{[7-(3,4-Difluoro-phenoxy)-4-hydroxy-isoquinoline-3-carbonyl]-amino}-propionic acid; 2-(S)-[(4-Hydroxy-7-phenylsulfanyl-isoquinoline-3-carbonyl)-amino]-propionic acid; 2-(R)-[(4-Hydroxy-7-phenylsulfanyl-isoquinoline-3-carbonyl)-amino]-propionic acid; 2-(R)-[(4-Hydroxy-7-phenoxy-isoquinoline-3-carbonyl)-amino]-propionic acid; 2-(S)-{[4-Hydroxy-7-(4-methoxy-phenoxy)-isoquinoline-3-carbonyl]-amino}-propionic acid; 2-(S)-[(7-Benzenesulfonyl-4-hydroxy-isoquinoline-3-carbonyl)-amino]-propionic acid; (R)-2-[(4-Hydroxy-1-methoxymethyl-7-phenoxy-isoquinoline-3-carbonyl)-amino]-propionic acid; (S)-2-[(4-Hydroxy-1-methoxymethyl-7-phenoxy-isoquinoline-3-carbonyl)-amino]-propionic acid; (S)-2-[(4-Mercapto-7-phenoxy-isoquinoline-3-carbonyl)-amino]-propionic acid; (S)-2-{[1-(4-Chloro-phenylsulfanyl)-4-hydroxy-isoquinoline-3-carbonyl]-amino}-propionic acid; (R)-2-{[1-(4-Chloro-phenylsulfanyl)-4-hydroxy-isoquinoline-3-carbonyl]-amino}-propionic acid; [(4-Hydroxy-7-phenylsulfanyl-isoquinoline-3-carbonyl)-amino]-acetic acid; [(4-Hydroxy-6-phenylsulfanyl-isoquinoline-3-carbonyl)-amino]-acetic acid; [(1-Chloro-4-hydroxy-7-phenylsulfanyl-isoquinoline-3-carbonyl)-amino]-acetic acid; [(1-Chloro-4-hydroxy-6-phenylsulfanyl-isoquinoline-3-carbonyl)-amino]-acetic acid; [(1-Bromo-4-hydroxy-7-phenylsulfanyl-isoquinoline-3-carbonyl)-amino]-acetic acid; [(1-Bromo-4-hydroxy-6-phenylsulfanyl-isoquinoline-3-carbonyl)-amino]-acetic acid; [(4-Hydroxy-7-phenoxy-isoquinoline-3-carbonyl)-amino]-acetic acid; [(4-Hydroxy-6-phenoxy-isoquinoline-3-carbonyl)-amino]-acetic acid; [(1-Chloro-4-hydroxy-7-phenoxy-isoquinoline-3-carbonyl)-amino]-acetic acid; [(1-Chloro-4-hydroxy-6-phenoxy-isoquinoline-3-carbonyl)-amino]-acetic acid; [(1-Bromo-4-hydroxy-7-phenoxy-isoquinoline-3-carbonyl)-amino]-acetic acid; [(1-Bromo-4-hydroxy-6-phenoxy-isoquinoline-3-carbonyl)-amino]-acetic acid; {[7-(2,6-Dimethyl-phenoxy)-4-hydroxy-isoquinoline-3-carbonyl]-amino}-acetic acid; {[1-Chloro-7-(2,6-dimethyl-phenoxy)-4-hydroxy-isoquinoline-3-carbonyl]-amino}-acetic acid; {[1-Bromo-7-(2,6-dimethyl-phenoxy)-4-hydroxy-isoquinoline-3-carbonyl]-amino}-acetic acid; [(1-Bromo-7-chloro-4-hydroxy-isoquinoline-3-carbonyl)-amino]-acetic acid; [(1-Bromo-6-chloro-4-hydroxy-isoquinoline-3-carbonyl)-amino]-acetic acid; [(1-Bromo-4-hydroxy-7-trifluoromethyl-isoquinoline-3-carbonyl)-amino]-acetic acid; [(1-Bromo-4-hydroxy-6-trifluoromethyl-isoquinoline-3-carbonyl)-amino]-acetic acid; [(4-Hydroxy-1-phenoxy-isoquinoline-3-carbonyl)-amino]-acetic acid; [(1,7-dibromo-4-hydroxy-isoquinoline-3-carbonyl)-amino]-acetic acid; [(7-Bromo-1-chloro-4-hydroxy-isoquinoline-3-carbonyl)-amino]-acetic acid; [(6-Bromo-4-hydroxy-isoquinoline-3-carbonyl)-amino]-acetic acid; [(1-Bromo-7-fluoro-4-hydroxy-isoquinoline-3-carbonyl)-amino]-acetic acid; [(7-Fluoro-4-hydroxy-isoquinoline-3-carbonyl)-amino]-acetic acid; [(1-Chloro-7-fluoro-4-hydroxy-isoquinoline-3-carbonyl)-amino]-acetic acid; [(1-Chloro-4-hydroxy-benzo[g]isoquinoline-3-carbonyl)-amino]-acetic acid; [(1-Bromo-4-hydroxy-isoquinoline-3-carbonyl)-amino]-acetic acid; [(4-Hydroxy-6-phenyl-isoquinoline-3-carbonyl)-amino]-acetic acid; [(4-Hydroxy-7-phenyl-isoquinoline-3-carbonyl)-amino]-acetic acid; [(1-Chloro-4-hydroxy-6-phenyl-isoquinoline-3-carbonyl)-amino]-acetic acid; [(1-Chloro-4-hydroxy-7-phenyl-isoquinoline-3-carbonyl)-amino]-acetic acid; [(1-Bromo-4-hydroxy-6-phenyl-isoquinoline-3-carbonyl)-amino]-acetic acid; [(1-Bromo-4-hydroxy-7-phenyl-isoquinoline-3-carbonyl)-amino]-acetic acid; [(4-Hydroxy-5-phenyl-isoquinoline-3-carbonyl)-amino]-acetic acid; [(4-Hydroxy-8-phenyl-isoquinoline-3-carbonyl)-amino]-acetic acid; [(1-Chloro-4-hydroxy-5-phenyl-isoquinoline-3-carbonyl)-amino]-acetic acid; [(1-Chloro-4-hydroxy-8-phenyl-isoquinoline-3-carbonyl)-amino]-acetic acid; [(1-Bromo-4-hydroxy-5-phenyl-isoquinoline-3-carbonyl)-amino]-acetic acid; [(1-Bromo-4-hydroxy-8-phenyl-isoquinoline-3-carbonyl)-amino]-acetic acid; [(1-Ethylsulfanyl-4-hydroxy-isoquinoline-3-carbonyl)-amino]-acetic acid; {[4-Hydroxy-1-(4-methoxy-phenylsulfanyl)-isoquinoline-3-carbonyl]-amino}-acetic acid; [(1-Chloro-4-hydroxy-7-iodo-isoquinoline-3-carbonyl)-amino]-acetic acid; [(1-Chloro-4-hydroxy-6-iodo-isoquinoline-3-carbonyl)-amino]-acetic acid; [(4-Hydroxy-7-iodo-isoquinoline-3-carbonyl)-amino]-acetic acid; [(4-Hydroxy-7-iodo-isoquinoline-3-carbonyl)-amino]-acetic acid; [(1-Bromo-7-butoxy-4-hydroxy-isoquinoline-3-carbonyl)-amino]-acetic acid; [(1-Bromo-6-butoxy-4-hydroxy-isoquinoline-3-carbonyl)-amino]-acetic acid; [(6-Benzyloxy-1-chloro-4-hydroxy-isoquinoline-3-carbonyl)-methyl-amino]-acetic acid; [(1-Chloro-4-hydroxy-isoquinoline-3-carbonyl)-methyl-amino]-acetic acid; [(1-Chloro-4-hydroxy-6-isopropoxy-isoquinoline-3-carbonyl)-methyl-amino]-acetic acid; [(1-Chloro-4-hydroxy-7-isopropoxy-isoquinoline-3-carbonyl)-methyl-amino]-acetic acid; [Carboxymethyl-(1-chloro-4-hydroxy-isoquinoline-3-carbonyl)-amino]-acetic acid; [Carboxymethyl-(1-chloro-4-hydroxy-6-isopropoxy-isoquinoline-3-carbonyl)-amino]-acetic acid; 1-Chloro-4-hydroxy-isoquinoline-3-carboxylic acid (2-amino-ethyl)-amide(trifluoro-acetic acid salt); 1-Chloro-4-hydroxy-isoquinoline-3-carboxylic acid (2-methoxy-ethyl)-amide; 1-Chloro-4-hydroxy-isoquinoline-3-carboxylic acid (2-hydroxy-ethyl)-amide; 1-Chloro-4-hydroxy-isoquinoline-3-carboxylic acid (2-dimethylamino-ethyl)-amide; 1-Chloro-4-hydroxy-isoquinoline-3-carboxylic acid (2-acetylamino-ethyl)-amide; 1-Chloro-4-hydroxy-6-isopropoxy-isoquinoline-3-carboxylic acid (2-hydroxy-ethyl)-amide; 1-Chloro-4-hydroxy-6-isopropoxy-isoquinoline-3-carboxylic acid (2-methoxy-ethyl)-amide; 1-Chloro-4-hydroxy-6-isopropoxy-isoquinoline-3-carboxylic acid (2-amino-ethyl)-amide (trifluoro-acetic acid salt); 1-Chloro-4-hydroxy-6-isopropoxy-isoquinoline-3-carboxylic acid (2-dimethylamino-ethyl)-amide; 1-Chloro-4-hydroxy-7-isopropoxy-isoquinoline-3-carboxylic acid (2-amino-ethyl)-amide (trifluoroacetic acid salt); 1-Chloro-4-hydroxy-7-isopropoxy-isoquinoline-3-carboxylic acid (2-methoxy-ethyl)-amide; 1-Chloro-4-hydroxy-7-isopropoxy-isoquinoline-3-carboxylic acid (2-dimethylamino-ethyl)-amide; 1-Chloro-4-hydroxy-7-isopropoxy-isoquinoline-3-carboxylic acid (2-hydroxy-ethyl)-amide; (S)-2-[(6-Benzyloxy-1-chloro-4-hydroxy-isoquinoline-3-carbonyl)-amino]-propionic acid; (R)-2-[(1-Chloro-4-hydroxy-isoquinoline-3-carbonyl)-amino]-3-hydroxy-propionic acid; (S)-2-[(1-Chloro-4-hydroxy-isoquinoline-3-carbonyl)-amino]-3-hydroxy-propionic acid; (R)-2-[(1-Chloro-4-hydroxy-6-isopropoxy-isoquinoline-3-carbonyl)-amino]-3-hydroxy-propionic acid; (S)-2-[(1-Chloro-4-hydroxy-6-isopropoxy-isoquinoline-3-carbonyl)-amino]-3-hydroxy-propionic acid; (R)-2-[(1-Chloro-4-hydroxy-7-isopropoxy-isoquinoline-3-carbonyl)-]-3-hydroxy-propionic acid; (S)-2-[(1-Chloro-4-hydroxy-7-isopropoxy-isoquinoline-3-carbonyl)-amino]-3-hydroxy-propionic acid; 2-[(1-Chloro-4-hydroxy-isoquinoline-3-carbonyl)-amino]-2-methyl-propionic acid; 2-[(1-Chloro-4-hydroxy-6-isopropoxy-isoquinoline-3-carbonyl)-amino]-2-methyl-propionic acid; (R)-2-[(1-Chloro-4-hydroxy-isoquinoline-3-carbonyl)-amino]-3-(1H-imidazol-4-yl)-propionic acid(trifluoro-acetic acid salt); (S)-2-[(1-Chloro-4-hydroxy-isoquinoline-3-carbonyl)-amino]-3-(1H-imidazol-4-yl)-propionic acid (trifluoro-acetic acid salt); (R)-2-[(1-Chloro-4-hydroxy-isoquinoline-3-carbonyl)-amino]-3-methyl-butyric acid; (S)-2-[(1-Chloro-4-hydroxy-isoquinoline-3-carbonyl)-amino]-3-methyl-butyric acid; (R)-2-[(1-Chloro-4-hydroxy-6-isopropoxy-isoquinoline-3-carbonyl)-amino]-3-methyl-butyric acid; (S)-2-[(1-Chloro-4-hydroxy-6-isopropoxy-isoquinoline-3-carbonyl)-amino]-3-methyl-butyric acid; (R)-2-[(1-Chloro-4-hydroxy-7-isopropoxy-isoquinoline-3-carbonyl)-amino]-3-methyl-butyric acid; (S)-2-[(1-Chloro-4-hydroxy-7-isopropoxy-isoquinoline-3-carbonyl)-amino]-3-methyl-butyric acid; (S)-2-[(6-Benzyloxy-1-chloro-4-hydroxy-isoquinoline-3-carbonyl)-amino]-3-methyl-butyric acid; (R)-2-[(1-Chloro-4-hydroxy-isoquinoline-3-carbonyl)-amino]-3-phenyl-propionic acid; (S)-2-[(1-Chloro-4-hydroxy-isoquinoline-3-carbonyl)-amino]-3-phenyl-propionic acid; (R)-2-[(1-Chloro-4-hydroxy-6-isopropoxy-isoquinoline-3-carbonyl)-amino]-3-phenyl-propionic acid; (S)-2-[(1-Chloro-4-hydroxy-6-isopropoxy-isoquinoline-3-carbonyl)-amino]-3-phenyl-propionic acid; (R)-2-[(1-Chloro-4-hydroxy-7-isopropoxy-isoquinoline-3-carbonyl)-amino]-3-phenyl-propionic acid; (S)-2-[(1-Chloro-4-hydroxy-7-isopropoxy-isoquinoline-3-carbonyl)-amino]-3-phenyl-propionic acid; (R)-2-[(1-Chloro-4-hydroxy-isoquinoline-3-carbonyl)-amino]-3-(4-hydroxy-phenyl)-propionic acid; (S)-2-[(1-Chloro-4-hydroxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-3-(4-hydroxy-phenyl)-propionic acid; (S)-2-[(1-Chloro-4-hydroxy-6-isopropoxy-isoquinoline-3-carbonyl)-amino]-3-(4-hydroxy-phenyl)-propionic acid; (R)-2-[(1-Chloro-4-hydroxy-7-isopropoxy-isoquinoline-3-carbonyl)-amino]-3-(4-hydroxy-phenyl)-propionic acid; (S)-2-[(1-Chloro-4-hydroxy-7-isopropoxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-pentanoic acid; (S)-2-[(1-Chloro-4-hydroxy-6-isopropoxy-isoquinoline-3-carbonyl)-amino]-pentanoic acid; (R)-1-(1-Chloro-4-hydroxy-isoquinoline-3-carbonyl)-pyrrolidine-2-carboxylic acid; (S)-1-(1-Chloro-4-hydroxy-isoquinoline-3-carbonyl)-pyrrolidine-2-carboxylic acid; (R)-1-(1-Chloro-4-hydroxy-6-isopropoxy-isoquinoline-3-carbonyl)-pyrrolidine-2-carboxylic acid; (S)-1-(1-Chloro-4-hydroxy-6-isopropoxy-isoquinoline-3-carbonyl)-pyrrolidine-2-carboxylic acid; (R)-6-Amino-2-[(1-chloro-4-hydroxy-isoquinoline-3-carbonyl)-amino]-hexanoic acid (trifluoro-acetic acid salt); (S)-6-Amino-2-[(1-chloro-4-hydroxy-isoquinoline-3-carbonyl)-amino]-hexanoic acid (trifluoro-acetic acid salt); (R)-6-Amino-2-[(1-chloro-4-hydroxy-6-isopropoxy-isoquinoline-3-carbonyl)-amino]-hexanoic acid; trifluoroacetic acid salt; (S)-6-Amino-2-[(1-chloro-4-hydroxy-6-isopropoxy-isoquinoline-3-carbonyl)-amino]-hexanoic acid (trifluoro-acetic acid salt); (R)-6-Amino-2-[(1-chloro-4-hydroxy-7-isopropoxy-isoquinoline-3-carbonyl)-amino]-hexanoic acid; trifluoroacetic acid salt; (S)-6-Amino-2-[(1-chloro-4-hydroxy-7-isopropoxy-isoquinoline-3-carbonyl)-amino]-hexanoic acid (trifluoro-acetic acid salt); (R)-2-[(1-Chloro-4-hydroxy-isoquinoline-3-carbonyl)-amino]-succinic acid; (S)-2-[(1-Chloro-4-hydroxy-isoquinoline-3-carbonyl)-amino]-succinic acid; (R)-2-[(1-Chloro-4-hydroxy-6-isopropoxy-isoquinoline-3-carbonyl)-amino]-succinic acid; (S)-2-[(1-Chloro-4-hydroxy-6-isopropoxy-isoquinoline-3-carbonyl)-amino]-succinic acid; (R)-2-[(1-Chloro-4-hydroxy-7-isopropoxy-isoquinoline-3-carbonyl)-amino]-succinic acid; 1-[(1-Chloro-4-hydroxy-isoquinoline-3-carbonyl)-amino]-cyclopropanecarboxylic acid; 1-[(1-Chloro-4-hydroxy-6-isopropoxy-isoquinoline-3-carbonyl)-amino]-cyclopropanecarboxylic acid; Dideutero-[(1-chloro-4-hydroxy-isoquinoline-3-carbonyl)-amino]-acetic acid; (R)-2-[(6-Benzyloxy-1-chloro-4-hydroxy-isoquinoline-3-carbonyl)-amino]-propionic acid; (S)-2-[(7-Benzyloxy-1-chloro-4-hydroxy-isoquinoline-3-carbonyl)-amino]-propionic acid; (R)-2-[(7-Benzyloxy-1-chloro-4-hydroxy-isoquinoline-3-carbonyl)-amino]-propionic acid; (S)-2-[(1-Chloro-4-hydroxy-isoquinoline-3-carbonyl)-amino]-propionic acid; (R)-2-[(1-Chloro-4-hydroxy-isoquinoline-3-carbonyl)-amino]-propionic acid; (S)-2-[(6-Isopropoxy-1-chloro-4-hydroxy-isoquinoline-3-carbonyl)-amino]-propionic acid; (R)-2-[6-Isopropoxy-1-chloro-4-hydroxy-isoquinoline-3-carbonyl)-amino]-propionic acid; (S)-2-[(7-Isopropoxy-1-chloro-4-hydroxy-isoquinoline-3-carbonyl)-amino-propionic acid; (R)-2-[(7-Isopropoxy-1-chloro-4-hydroxy-isoquinoline-3-carbonyl)-amino]-propionic acid; 1-Chloro-4-hydroxy-6-isopropoxy-isoquinoline-3-carboxylic acid (2-hydroxy-1-hydroxymethyl-ethyl)-amide; 1-Chloro-4-hydroxy-7-isopropoxy-isoquinoline-3-carboxylic acid (2-hydroxy-1-hydroxymethyl-ethyl)-amide; 1-Chloro-4-hydroxy-isoquinoline-3-carboxylic acid (2-hydroxy-1-hydroxymethyl-ethyl)-amide; {[7-(3,5-Difluoro-phenoxy)-4-hydroxy-isoquinoline-3-carbonyl]-amino}-acetic acid; {[6-(3,5-Difluoro-phenoxy)-4-hydroxy-isoquinoline-3-carbonyl]-amino}-acetic acid; ({7-[4-(4-Fluoro-phenoxy)-phenoxy]-4-hydroxy-isoquinoline-3-carbonyl}-amino)-acetic acid; ({6-[4-(4-Fluoro-phenoxy)-phenoxy]-4-hydroxy-isoquinoline-3-carbonyl}-amino)-acetic acid; {[7-(3-Chloro-4-fluoro-phenoxy)-4-hydroxy-isoquinoline-3-carbonyl]-amino}-acetic acid; {[6-(3-Chloro-4-fluoro-phenoxy)-4-hydroxy-isoquinoline-3-carbonyl]-amino}-acetic acid; (S)-2-{[7-(3-Fluoro-5-methoxy-phenoxy)-4-hydroxy-isoquinoline-3-carbonyl]-amino}-propionic acid; 2-(S)-[(7-Cyclohexyloxy-4-hydroxy-isoquinoline-3-carbonyl)-amino]-propionic acid; 2-(S)-[7-(4-Fluoro-phenoxy)-4-hydroxy-1-methyl-isoquinoline-3-carbonyl]-amino) propionic acid; 2-(S)-{[7-(4-Fluoro-phenoxy)-4-hydroxy-isoquinoline-3-carbonyl]-amino}-propionic acid; 2-(S)-[(4-Hydroxy-1-methyl-7-phenoxy-isoquinoline-3-carbonyl)-amino]-propionic acid; 2-(S)-[(4-Hydroxy-1-methyl-7-phenylsulfanyl-isoquinoline-3-carbonyl)-amino]-propionic acid; 2-(S)-{[4-Hydroxy-7-(4-trifluoromethyl-phenoxy)-isoquinoline-3-carbonyl]-amino}-propionic acid; {[7-(4-Chloro-phenoxy)-4-hydroxy-1-methyl-isoquinoline-3-carbonyl]-amino}-acetic acid; {[6-(4-Chloro-phenoxy)-4-hydroxy-1-methyl-isoquinoline-3-carbonyl]-amino}-acetic acid; {[7-(3,5-Difluoro-phenoxy)-4-hydroxy-1-methyl-isoquinoline-3-carbonyl]-amino}-acetic acid; {[4-Hydroxy-7-(4-methoxy-phenoxy)-1-methyl-isoquinoline-3-carbonyl]-amino}-acetic acid; {[4-Hydroxy-6-(4-methoxy-phenoxy)-1-methyl-isoquinoline-3-carbonyl]-amino}-acetic acid; [(6-Cyclohexyloxy-4-hydroxy-isoquinoline-3-carbonyl)-amino]-acetic acid; [(7-Cyclohexyloxy-4-hydroxy-isoquinoline-3-carbonyl)-amino]-acetic acid; [(7-Cyclohexyloxy-4-hydroxy-1-methyl-isoquinoline-3-carbonyl)-amino]-acetic acid; [(7-Cyclohexylsulfanyl-4-hydroxy-isoquinoline-3-carbonyl)-amino]-acetic acid; [(7-Cyclohexanesulfonyl-4-hydroxy-isoquinoline-3-carbonyl)-amino]-acetic acid; [(4-Hydroxy-1-isobutyl-isoquinoline-3-carbonyl)-amino]-acetic acid; [(4-Hydroxy-1-pyridin-2-yl-isoquinoline-3-carbonyl)-amino]-acetic acid; [(1-Ethyl-4-hydroxy-7-phenoxy-isoquinoline-3-carbonyl)-amino]-acetic acid; [(1-Dimethylaminomethyl-4-hydroxy-7-phenylsulfanyl-isoquinoline-3-carbonyl)-amino]-acetic acid; [(4-Hydroxy-1-methyl-7-phenylsulfanyl-isoquinoline-3-carbonyl)-amino]-acetic acid; {[4-Hydroxy-1-methyl-7-(4-trifluoromethyl-phenoxy)-isoquinoline-3-carbonyl]-amino}-acetic acid; or pharmaceutically acceptable salts, esters, or prodrugs thereof.

18. The method of claim 11, wherein the nitrogen-containing heteroaryl compound is represented by formula II: 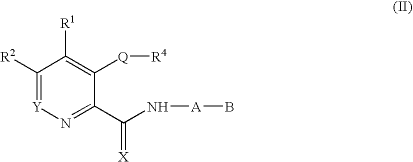
wherein
A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer.
B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO₂, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di-(C₁-C₁₂)-alkylcarbamoyl, N—(C₃-C₈)-cycloalkylcarbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₆)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N-((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C₇-C₁₆)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino-(C₁-C₈)-alkyl, amino-(C₁-C₁₀)-alkyl, N—(C₁-C₁₀)-alkylamino-(C₁-C₁₀)-alkyl, N.N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N.N-di-(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₁-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N—(C₆-C₁₂)-arylcarbamoyl, N—(C₇-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₆)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, N.N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;
X is O or S;
Q is O, S, NR′, or a bond;
where, if Q is a bond, R⁴ is halogen, nitrile, or trifluoromethyl;
or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C_{f}H_{(2f+1-g)}—F_{g}—, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁)-aralkyl radical, or a radical of the formula Z
—[CH₂]ᵥ—[O]_{w}—[CH₂]ₜ-E (Z)
where
E is a heteroaryl radical, a (C₃-C₈)-cycloalkyl radical, or a phenyl radical of the formula F 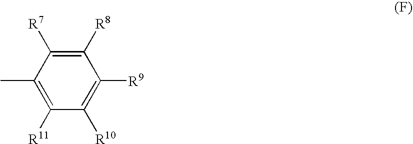
v is 0-6,
w is 0 or 1,
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R_{Y} and R_{Z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)-aralkoxy, (C₁-C₁₂)-alkylcarbonyl, (C₃-C₈)-cycloalkylcarbonyl, (C₆-C₁₂)-arylcarbonyl, (C₇-C₁₆)-aralkylcarbonyl; or
further wherein R^{Y} and R^{Z} together are —[CH₂]ₕ, in which a CH₂ group can be replaced by O, S, N—(C₁-C₄)-alkylcarbonylimino, or N—(C₁-C₄)-alkoxycarbonylimino, and h is 3 to 7; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷-R¹¹, or if E is a cycloalkyl radical, the radical can carry one substituent selected from those defined for R⁷—R″;
or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted (C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7.
Y is N or CR³;
R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓC_{f}H_{(2f+1-g)}—F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₈)-alkoxy-(C₁-C₁₀)-alkyl)-carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R 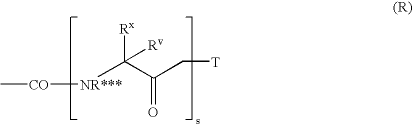
in which
R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,
s is 1-5,
T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7;
carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyl-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₆)-aralkyloxy-(C₁-C₁₀)-alkyl)-carbamoyl, CON(CH₂)ₕ, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—-(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy, N—(C₁-C₁₀)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy, N—(C₁-C₁₀)-alkyl-N—((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]ₒ, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;
or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae IIa, IIb and IIc: 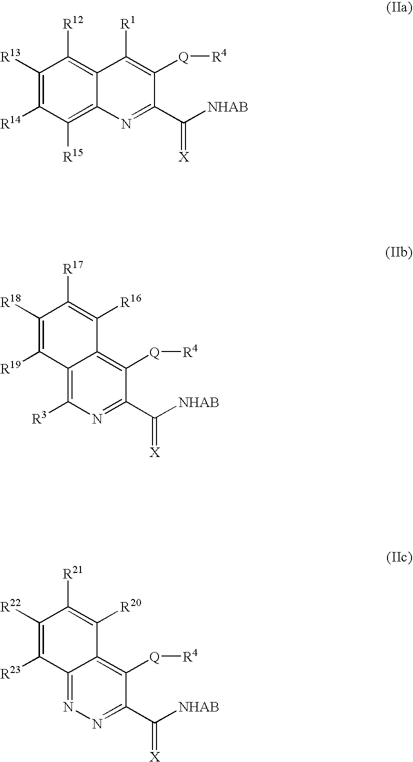
and the substituents R¹² to R²³ in each case independently of each other have the meaning of R¹, R² and R³;
or wherein the radicals R¹ and R², together with the pyridine carrying them, form a compound of Formula IId: 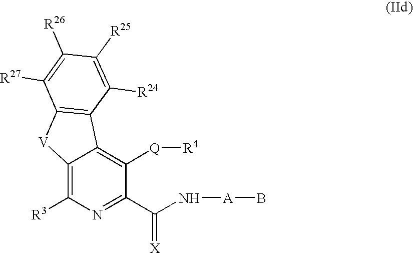
where V is S, O, or NR^{k}, and R^{k} is selected from hydrogen, (C₁-C₆)-alkyl, aryl, or benzyl;
where an aryl radical may be optionally substituted by 1 to 5 substituents as defined above; and
R²⁴, R²⁵, R²⁶, and R²⁷ in each case independently of each other have the meaning of R¹, R² and R³;
f is 1 to 8;
g is 0 or 1 to (2f+1);
x is 0 to 3; and
h is 3 to 7;
including the physiologically active salts and prodrugs derived therefrom.

19. The method of claim 11, wherein the nitrogen-containing heteroaryl compound is represented by formula III: 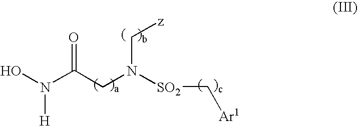
or pharmaceutically acceptable salts thereof, wherein:
a is an integer from 1 to 4;
b is an integer from 0 to 4;
c is an integer from 0 to 4;
Z is selected from the group consisting of (C₃-C₁₀)-cycloalkyl, (C₃-C₁₀)-cycloalkyl independently substituted with one or more Y¹, 3-10 membered heterocycloalkyl and 3-10 membered heterocycloalkyl independently substituted with one or more Y¹; (C₅-C₂₀)-aryl, (C₅-C₂₀)-aryl independently substituted with one or more Y¹, 5-20 membered heteroaryl and 5-20 membered heteroaryl independently substituted with one or more Ar¹ is selected from the group consisting of (C₅-C₂₀)-aryl, (C₅-C₂₀) aryl independently substituted with one or more Y², 5-20 membered heteroaryl and 5-20 membered heteroaryl independently substituted with one or more Y²;
each Y¹ is independently selected from the group consisting of a lipophilic functional group, (C₅-C₂₀)-aryl, (C₆-C₂₆)-alkaryl, 5-20 membered heteroaryl and 6-26 membered alk-heteroaryl;
each Y² is independently selected from the group consisting of —R′, —OR′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′)═NOR′, —NR′—C(O)R′, —SO₂R′, —SO₂R″, —NR′—SO₂—R′, —NR′—C(O)—NR′R′, tetrazol-5-yl, —NR′—C(O)—OR′, —C(NR′R′)═NR′, —S(O)—R′, S(O)—R″, and —NR′—C(S)—NR′R′; and
each R′ is independently selected from the group consisting of —H, (C₁-C₈)-alkyl, (C₂-C₈)-alkenyl, and (C₂-C₈)-alkynyl; and
each R″ is independently selected from the group consisting of (C₅-C₂₀)-aryl and (C₅-C₂₀)-aryl independently substituted with one or more —OR′, —SR′, —NR′R′, —NO₂, —CN, halogen or trihalomethyl groups, or wherein c is 0 and Ar¹ is an N′ substituted urea-aryl, the compound has the structural formula (IIIa): 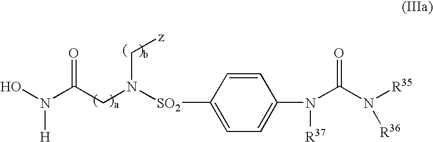
or pharmaceutically acceptable salts thereof, wherein:
a, b, and Z are as defined above; and
R³⁵ and R³⁶ are each independently selected from the group consisting of hydrogen, (C₁-C₈)-alkyl, (C₂-C₈)-alkenyl, (C₂-C₈)-alkynyl, (C₃-C₁₀)-cycloalkyl, (C₅-C₂₀)-aryl, (C₅-C₂₀)-substituted aryl, (C₆-C₂₆)-alkaryl, (C₆-C₂₆)-substituted alkaryl, 5-20 membered heteroaryl, 5-20 membered substituted heteroaryl, 6-26 membered alk-heteroaryl, and 6-26 membered substituted alk-heteroaryl; and
R³⁷ is independently selected from the group consisting of hydrogen, (C₁-C₈)-alkyl, (C₂-C₈)-alkenyl, and (C₂-C₈)-alkynyl.

20. The method of claim 11, wherein the nitrogen-containing heteroaryl compound is represented by formula IV: 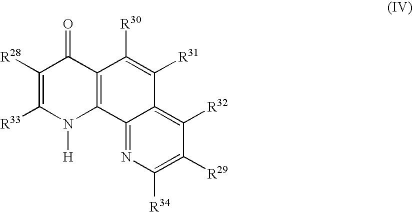
wherein
R²⁸ is hydrogen, nitro, amino, cyano, halogen, (C₁-C₄)-alkyl, carboxy or a metabolically labile ester derivative thereof; (C₁-C₄)-alkylamino, di-(C₁-C₄)-alkylamino, (C₁-C₆)-alkoxycarbonyl, (C₂-C₄)-alkanoyl, hydroxy-(C₁-C₄)-alkyl, carbamoyl, N—(C₁-C₄)-alkylcarbamoyl, (C₁-C₄)-alkylthio, (C₁-C₄)-alkylsulfinyl, (C₁-C₄)-alkylsulfonyl, phenylthio, phenylsulfinyl, phenylsulfonyl, said phenyl or phenyl groups being optionally substituted with 1 to 4 identical or different halogen, (C₁-C₄)-alkyoxy, (C₁-C₄)-alkyl, cyano, hydroxy, trifluoromethyl, fluoro-(C₁-C₄)-alkylthio, fluoro-(C₁-C₄)-alkylsulfinyl, fluoro-(C₁-C₄)-alkylsulfonyl, (C₁-C₄)-alkoxy-(C₂-C₄)-alkoxycarbonyl, N,N-di-[(C₁-C₄)-alkyl]carbamoyl-(C₁-C₄)-alkoxycarbonyl, (C₁-C₄)-alkylamino-(C₂-C₄)-alkoxycarbonyl, di-(C₁-C₄)-alkylamino-(C₂-C₄)-alkoxycarbonyl, (C₁-C₄)-alkoxy-(C₂-C₄)-alkoxy-(C₂-C₄)-alkoxycarbonyl, (C₂-C₄)-alkanoyloxy-(C₁-C₄)-alkyl, or N-[amino-(C₂-C₈)-alkyl]-carbamoyl;
R²⁹ is hydrogen, hydroxy, amino, cyano, halogen, (C₁-C₄)-alkyl, carboxy or metabolically labile ester derivative thereof, (C₁-C₄)-alkylamino, di-(C₁-C₄)-alkylamino, (C₁-C₆)-alkoxycarbonyl, (C₂-C₄)-alkanoyl, (C₁-C₄)-alkoxy, carboxy-(C₁-C₄)-alkoxy, (C₁-C₄)-alkoxycarbonyl-(C₁-C₄)-alkoxy, carbamoyl, N—(C₁-C₈)-alkylcarbamoyl, N,N-di-(C₁-C₈)-alkylcarbamoyl, N-[amino-(C₂-C₈)-alkyl]-carbamoyl, N—[(C₁-C₄)-alkylamino-(C₁-C₈)-alkyl]-carbamoyl, N-[di-(C₁-C₄)-alkylamino-(C₁-C₈)-alkyl)]-carbamoyl, N-cyclohexylcarbamoyl, N-[cyclopentyl]-carbamoyl, N—(C₁-C₄)-alkylcyclohexylcarbamoyl, N—(C₁-C₄)-alkylcyclopentylcarbamoyl, N-phenylcarbamoyl, N—(C₁-C₄)-alkyl-N-phenylcarbamoyl, N,N-diphenylcarbamoyl, N-[phenyl-(C₁-C₄)-alkyl]-carbamoyl, N—(C₁-C₄)-alkyl-N-[phenyl-(C₁-C₄)-alkyl]-carbamoyl, or N,N-di-[phenyl-(C₁-C₄)-alkyl]-carbamoyl, said phenyl or phenyl groups being optionally substituted with 1 to 4 identical or different halogen, (C₁-C₄)-alkyoxy, (C₁-C₄)-alkyl, cyano, hydroxy, trifluoromethyl, N—[(C₂-C₄)-alkanoyl]-carbamoyl, N—[(C₁-C₄)-alkoxycarbonyl]-carbamoyl, N-[fluoro-(C₂-C₆)-alkyl]-carbamoyl, N,N-[fluoro-(C₂-C₆)-alkyl]-N—(C₁-C₄)-alkylcarbamoyl, N,N-[di-fluoro-(C₂-C₆)-alkyl]carbamoyl, pyrrolidin-1-ylcarbonyl, piperidinocarbonyl, piperazin-1-ylcarbonyl, morpholinocarbonyl, wherein the heterocyclic group, is optionally substituted with 1 to 4, (C₁-C₄)-alkyl, benzyl, 1,2,3,4-tetrahydro-isoquinolin-2-ylcarbonyl, N,N-[di-(C₁-C₄)-alkyl]-thiocarbamoyl, N—(C₂-C₄)-alkanoylamino, or N—[(C₁-C₄)-alkoxycarbonyl]-amino;
R³⁰ is hydrogen, (C₁-C₄)-alkyl, (C₂-C₄)-alkoxy, halo, nitro, hydroxy, fluoro-(C₁-C₄)-alkyl, or pyridinyl;
R³¹ is hydrogen, (C₁-C₄)-alkyl, (C₂-C₄)-alkoxy, halo, nitro, hydroxy, fluoro-(C₁-C₄)-alkyl, pyridinyl, or methoxy;
R³² is hydrogen, hydroxy, amino, (C₁-C₄)-alkylamino, di-(C₁-C₄)-alkylamino, halo, (C₁-C₄)-alkoxy-(C₂-C₄)-alkoxy, fluoro-(C₁-C₆)-alkoxy, pyrrolidin-1-yl, piperidino, piperazin-1-yl, or morpholino, wherein the heterocyclic group is optionally substituted with 1 to 4 identical or different (C₁-C₄)-alkyl or benzyl; and
R³³ and R³⁴ are individually selected from hydrogen, (C₁-C₄)-alkyl, and (C₁-C₄)-alkoxy;
including pharmaceutically-acceptable salts and pro-drugs derived therefrom.

21. The method of claim 11, wherein the nitrogen-containing heteroaryl compound is selected from [(7-chloro-3-hydroxy-quinoline-2-carbonyl)-amino]-acetic acid; [(1-chloro-4-hydroxy-isoquinoline-3-carbonyl)-amino]-acetic acid; 4-oxo-1,4-dihydro-[1,10]phenathroline-3-carboxylic acid, [(3-hydroxy-6-isopropoxy-quinoline-2-carbonyl)-amino]-acetic acid; [(1-bromo-4-hydroxy-7-trifluoromethyl-isoquinoline-3-carbonyl)-amino]-acetic acid; 4-hydroxy-5-methoxy-[1,10]phenathroline-3-carboxylic acid ethyl ether; [(7-chloro-3-hydroxy-quinoline-2-carbonyl)-amino]-acetic acid, sodium salt; 3-{[4-(3,3-dibenzyl-ureido)-benzenesulfonyl]-[2-(4-methoxy-phenyl)-ethyl]-amino}-N-hydroxy-propionamide; [(4-hydroxy-7-phenylsulfanyl-isoquinoline-3-carbonyl)-amino]-acetic acid; [(4-hydroxy-6-phenylsulfanyl-isoquinoline-3-carbonyl)-amino]-acetic acid; [1-chloro-4-hydroxy-7-phenoxy-isoquinoline-3-carbonyl)-amino]-acetic acid; [(1-bromo-4-hydroxy-7-phenoxy-isoquinoline-3-carbonyl)-amino]-acetic acid; [(4-hydroxy-7-phenoxy-isoquinoline-3-carbonyl)-amino]-acetic acid; [(1-chloro-4-hydroxy-isoquinoline-3-carbonyl)-amino]-acetic acid; 3-carboxy-5-hydroxy-4-oxo-3,4-dihydro-phenathroline; 3-carboxy-5-methoxy-4-oxo-3,4-dihydro-1,10-phenathroline; 5-methoxy-4-oxo-1,4-dihydro-[1,10]phenathroline-3-carboxylic acid ethyl ester; 5-methoxy-4-oxo-1,4-dihydro-[1,10]phenanthroline-3-carboxylic acid; 3-carboxy-8-hydroxy-4-oxo-3,4-dihydro-1,10-phenanthroline; [(3-hydroxy-pyridine-2-carbonyl)-amino]-acetic acid; [(3-methoxy-pyridine-2-carbonyl)-amino]-acetic acid; 3-methoxypyridine-2-carboxylic acid N-(((hexadecyloxy)-carbonyl)-methyl)-amide hydrochloride; 3-methoxypyridine-2-carboxylic acid N-(((1-octyloxy)-carbonyl)-methyl)-amide; 3-methoxypyridine-2-carboxylic acid N-(((hexyloxy)-carbonyl)-methyl)-amide; 3-methoxypyridine-2-carboxylic acid N-(((butyloxy)-carbonyl)-methyl)-amide; 3-methoxypyridine-2-carboxylic acid N-(((2-nonyloxy)-carbonyl)-methyl)-amide racemate; 3-methoxypyridine-2-carboxylic acid N-(((heptyloxy)-carbonyl)-methyl)-amide; 3-benzyloxypyridine-2-carboxylic acid N-(((octyloxy)-carbonyl)-methyl)-amide; 3-benzyloxypyridine-2-carboxylic acid N-(((butyloxy)-carbonyl)-methyl)-amide; 5-(((3-(1-butyloxy)-propyl)-amino)-carbonyl)-3-methoxypyridine-2-carboxylic acid N-((benzyloxycarbonyl)-methyl)-amide; 5-(((3-(1-butyloxy)-propyl)-amino)-carbonyl)-3-methoxypyridine-2-carboxylic acid N-(((1-butyloxy)-carbonyl)-methyl)-amide; 5-(((3-lauryloxy)-propyl)amino)-carbonyl)-3-methoxypyridine-2-carboxylic acid N-(((benzyloxy)-carbonyl)-methyl)-amide; N-((6-(1-butyloxy)-3-hydroxyquinolin-2-yl)-carbonyl)-glycine; [(3-hydroxy-6-trifluoromethoxy-quinoline-2-carbonyl)-amino]-acetic acid; N-((6-chloro-3-hydroxyquinolin-2-yl)-carbonyl)-glycine; N-((7-chloro-3-hydroxyquinolin-2-yl)-carbonyl)-glycine; [(6-chloro-3-hydroxy-quinoline-2-carbonyl)-amino]-acetic acid; N-((1-chloro-4-hydroxy-7-(2-propyloxy)isoquinolin-3-yl)-carbonyl)-glycine; N-((1-chloro-4-hydroxy-6-(2-propyloxy)isoquinolin-3-yl)-carbonyl)-glycine; N-((1-chloro-4-hydroxy-isoquinoline-3-carbonyl)-amino)-acetic acid; N-((1-chloro-4-hydroxy-7-methoxyisoquinolin-3-yl)-carbonyl)-glycine; N-((1-chloro-4-hydroxy-6-methoxyisoquinolin-3-yl)-carbonyl)-glycine; N-((7-butyloxy)-1-chloro-4-hydroxyisoquinolin-3-yl)-carbonyl)-glycine; N-((6-benzyloxy-1-chloro-4-hydroxy-isoquinoline-3-carbonyl)-amino)-acetic acid; ((7-benzyloxy-1-chloro-4-hydroxy-isoquinoline-3-carbonyl)-amino)-acetic acid methyl ester; N-((7-benzyloxy-1-chloro-4-hydroxy-isoquinoline-3-carbonyl)-amino)-acetic acid; N-((8-chloro-4-hydroxyisoquinolin-3-yl)-carbonyl)-glycine; N-((7-butoxy-4-hydroxy-isoquinoline-3-carbonyl)-amino)-acetic acid; 6-cyclohexyl-1-hydroxy-4-methyl-1H-pyridin-2-one; 7-(4-methyl-piperazin-1-ylmethyl)-5-phenylsulfanylmethyl-quinolin-8-ol; 4-nitro-quinolin-8-ol; 5-butoxymethyl-quinolin-8-ol; 3-{{4-[3-(4-chloro-phenyl)-ureido]-benzenesulfonyl}-[2-(4-methoxy-phenyl)-ethyl]-amino}-N-hydroxy-propionamide; 3-{{4-[3-(1,2-diphenyl-ethyl)-ureido]-benzenesulfonyl}-[2-(4-methoxy-phenyl)-ethyl]-amino}-N-hydroxy-propionamide; or pharmaceutically acceptable salts; esters; or prodrugs thereof.

22. The method of claim 7, wherein the FIH inhibitor is an alanine derivative.

23. The method of claim 22, wherein the alanine derivative is N-oxalyl-D-alanine.

24. The method of claim 7, wherein the FIH inhibitor is a dihydroxybenzoic acid.

25. The method of claim 24, wherein the dihydroxybenzoic acid is 3,4-dihydroxybenzoate.

26. The method of claim 3, wherein the effector molecule is an siRNA which targets a PHD or FIH.

27. The method of claim 3, wherein the effector molecule is a NODDD or CODDD peptide.

28. The method of claim 27, wherein the NODDD peptide comprises the amino acid sequence of SEQ ID NO:2.

29. The method of claim 27, wherein the CODDD peptide comprises the amino acid sequence of SEQ ID NO:3.

30. The method of claim 1, wherein the effector molecule is betacellulin protein, a betacellulin protein variant, or a derivative thereof.

31. The method of claim 1, wherein the effector molecule is a betacellulin peptide.

32. The method of claim 31, wherein the effector molecule comprises the amino acid sequence of SEQ ID NO:1.

33. The method of claim 1, wherein the effector molecule is an antibody to betacellulin receptors.

34. The method of claim 1, wherein the effector molecule is a small molecule agonist of betacellulin receptors.

35. The method of claim 1, wherein the mammal is a human.

36. A method of screening for effector molecules which ameliorate vulvovaginal atrophy comprising:
(a) administering estrogen to a test subject;
(b) isolating mRNA from the vaginal cells of the test subject;
(c) comparing mRNA expression levels between the mRNA isolated in step (b) with mRNA isolated from a control subject;
(d) identifying a target mRNA based on the comparison of step (c); and
(e) identifying an effector molecule which modulates the activity of the target mRNA of step (d).

37. The method of claim 36, wherein step (b) is performed at least 3 hours after step (a).

38. The method of claim 36, wherein the test subject is a rat.

39. The method of claim 36, wherein the test subject is a human.

40. The method of claim 36, wherein the estrogen is 17β-estradiol.

41. The method of claim 36, wherein the estrogen is administered to the test subject in a dosage range of from about 0.1 μg to about 100 mg.

42. The method of claim 36, wherein the target mRNA encodes HIF1α protein, betacellulin protein, or OGFr protein.

43. The method of claim 36, wherein step (c) is performed by mRNA expression profiling.

44. The method of claim 36, wherein the target mRNA of step (d) is identified as having been detected in at least 10% of samples from the test subject or the control subject, has a fold change in mRNA expression level between the control subject and the test subject of at least 1.3, and the fold change has a p-value based on a T-test, ANOVA, Mann-Whitney Test, or Median Test of ≦0.05.

45. The method of claim 44, wherein the target mRNA of step (d) is identified as having been detected in at least 25% of samples from the test subject or the control subject.

46. The method of claim 44, wherein the target mRNA of step (d) has a fold change in mRNA expression-level between the control subject and the test subject of at least 1.7.

47. The method of claim 44, wherein the fold change has a p-value based on a T-test of ≦0.01.

48. An effector molecule identified by any of claims 3647.

49. A method of treating vulvovaginal atrophy comprising:
(a) administering estrogen to a test subject;
(b) isolating mRNA from the vaginal cells of the test subject;
(c) comparing mRNA expression levels between the mRNA isolated in step (b) with mRNA isolated from a control subject;
(d) identifying a target mRNA based on the comparison of step (c);
(e) identifying an effector molecule which modulates the activity of the target mRNA of step (d); and
(f) administering a therapeutically effective amount of the effector molecule of step (e) to a mammal in need of vulvovaginal atrophy treatment.

50. A method of identifying target mRNA which is regulated by estrogen comprising:
(a) administering estrogen to a test subject;
(b) isolating mRNA from the vaginal cells of the test subject;
(c) comparing mRNA expression levels between the mRNA isolated in step (b) with mRNA isolated from a control subject; and
(d) identifying a target mRNA based on the comparison of step (c).

51. The method of claim 50, wherein step (b) is performed at least 3 hours after step (a).

52. The method of claim 50, wherein the test subject is a rat.

53. The method of claim 50, wherein the test subject is a human.

54. The method of claim 50, wherein the estrogen is 17β-estradiol.

55. The method of claim 50, wherein the estrogen is administered to the test subject in a dosage range of from about 0.1 μg to about 100 mg.

56. The method of claim 50, wherein step (c) is performed by mRNA expression profiling.

57. The method of claim 50, wherein the target mRNA of step (d) is identified as having been detected in at least 10% of samples from the test subject or the control subject, has a fold change in mRNA expression level between the control subject and the test subject of at least 1.3, and the fold change has a p-value based on a T-test, ANOVA, Mann-Whitney Test, or Median Test of ≦0.05.

58. The method of claim 57, wherein the target mRNA of step (d) is identified as having been detected in at least 25% of samples from the test subject or the control subject.

59. The method of claim 57, wherein the target mRNA of step (d) has a fold change in mRNA expression level between the control subject and the test subject of at least 1.7.

60. The method of claim 57, wherein the fold change has a p-value based on a T-test of ≦0.01.

61. A target mRNA identified by the method of any of claims 50-60.
